(19) United States
(12) Patent Application Publication
(10) Pub. No.: US 2023/0270821 A1
(43) Pub. Date: Aug. 31, 2023

(54) FORMULATIONS COMPRISING ACTRII POLYPEPTIDE VARIANTS

(71) Applicant: Acceleron Pharma Inc., Cambridge, MA (US)

(72) Inventor: Robert Steininger, Cambridge, MA (US)

(21) Appl. No.: 18/035,329

(22) PCT Filed: Nov. 4, 2021
(86) PCT No.: PCT/US2021/058068

§ 371 Date: May 4, 2023

Related U.S. Application Data
(60) Provisional application No. 63/110,844, filed on Nov. 6, 2020.

(51) Int. Cl. A61K 38/17; A61K 47/26; A61K 47/12; A61K 9/19

(52) U.S. Cl. CPC A61K 38/179; A61K 47/26; A61K 47/12; A61K 9/19

(57) ABSTRACT

In certain aspects, the present disclosure provides dosing regimens, dosing forms, and formulations comprising a recombinant fusion protein comprising human activin receptor type-II (ActRII) polypeptides or derivatives thereof linked to a constant domain of an immunoglobulin, such as human IgG1 Fc domain. The disclosure also provides kits and methods for using such formulations to treat a human subject with a thalassemia or myelodysplastic syndromes (MDS).

### CROSS-REFERENCE TO RELATED APPLICATION

This application claims the benefit of priority from the U.S. Provisional Application No. 63/110,844, filed Nov. 6, 2020. The specification of the foregoing application is incorporated herein by reference in its entirety.

### BACKGROUND

The mature red blood cell, or erythrocyte, is responsible for oxygen transport in the circulatory systems of vertebrates. Red blood cells contain high concentrations of hemoglobin, a protein that binds oxygen in the lungs at relatively high partial pressure of oxygen (pO₂) and delivers oxygen to areas of the body with a relatively low pO₂.

Mature red blood cells are produced from pluripotent hematopoietic stem cells in a process termed crythropoiesis. Postnatal crythropoiesis occurs primarily in the bone marrow and in the red pulp of the spleen. The coordinated action of various signaling pathways control the balance of cell proliferation, differentiation, survival and death. Under normal conditions, red blood cells are produced at a rate that maintains a constant red cell mass in the body, and production may increase or decrease in response to various stimuli, including increased or decreased oxygen tension or tissue demand. The process of erythropoiesis begins with the formation of lineage committed precursor cells and proceeds through a series of distinct precursor cell types. The final stages of erythropoiesis occur as reticulocytes are released into the bloodstream and lose their mitochondria and ribosomes while assuming the morphology of mature red blood cell. An elevated level of reticulocytes, or an elevated reticulocyte:erythrocyte ratio, in the blood is indicative of increased red blood cell production rates.

Anemia is a broadly-defined condition characterized by lower than normal levels of hemoglobin or red blood cells in the blood. In some instances, anemia is caused by a primary disorder in the production or survival of red blood cells (e.g., a thalassemia disorder). More commonly, anemia is secondary to diseases of other systems [see, e.g., Weatherall & Provan (2000) Lancet 355, 1169-1175]. Anemia may result from a reduced rate of production or increased rate of destruction of red blood cells or by loss of red blood cells due to bleeding. Anemia may result from a variety of disorders that include, for example, acute or chronic renal failure or end stage renal disease, chemotherapy treatment, a myelodysplastic syndrome, rheumatoid arthritis, and bone marrow transplantation.

There is high unmet need for effective therapies for such red blood cell disorders. For example, sideroblastic anemia, which occurs in both inherited and acquired forms, is characterized by the presence of “ring sideroblasts” in bone marrow. These distinctive red blood cell precursors (erythroblasts) can be identified by the presence of perinuclear siderotic granules, which are revealed by histologic staining with Prussian blue and are indicative of pathologic iron deposits in mitochondria [see, e.g., Mufti et al. (2008) Haematologica 93:1712-1717; Bottomley et al. (2014) Hematol Oncol Clin N Am 28:653-670]. Acquired sideroblastic anemia occurs most frequently in the context of myelodysplastic syndromes. Endogenous EPO levels are commonly elevated in subsets of patients with MDS, thus suggesting that EPO has diminished effectiveness in these patients. It has been estimated that fewer than 10% of patients with MDS respond favorably to EPO [Estey (2003) Curr Opin Hematol 10, 60-67], while a more recent meta-analysis found that EPO response rates range from 30% to 60% depending on the study [Moyo et al (2008) Ann Hematol 87:527-536]. Compared to other MDS patients, those with ring sideroblasts tend to be at substantially lower risk of developing acute myeloid leukemia and would therefore stand to benefit for an extended period from anti-anemia therapeutic agents that do not contribute to systemic iron burden and that instead help to reduce the iron overload frequently present in such patients [see, e.g., Temraz et al., 2014, Crit Rev Oncol Hematol 91:64-73].

Thus, it is an object of the present disclosure to provide dosing regimens, dosage forms, and formulations comprising ActRII polypeptides and corresponding methods for treatment of subjects with anemia resulting from such red blood cell disorders, specifically thalassemias and MDS.

### SUMMARY

Provided herein are dosing regimens, dosage forms, and formulations comprising a recombinant fusion protein comprising an extracellular domain (ECD) of human activin receptor type-II (ActRII) polypeptides or derivatives thereof linked to a constant domain of an immunoglobulin, such as human IgG1 Fc domain. In certain aspects, the disclosure provides a dosing regimen for the treatment of thalassemia in a subject in need thereof comprising administering a lyophilized human ActRII polypeptide linked to a constant domain of an immunoglobulin, wherein the dosing regimen comprises: 1) administering an initial dose of 1 mg/kg: 2) monitoring a subject's response; and 3) modifying the subsequent dose; and wherein the subject is administered the subsequent dose every three weeks. In certain aspects, the disclosure provides a dosing regimen for the treatment of myelodysplastic syndrome in a subject in need thereof comprising administering a lyophilized human ActRII polypeptide linked to a constant domain of an immunoglobulin, wherein the dosing regimen comprises: 1) administering an initial dose of 1 mg/kg; 2) monitoring a subject's response; and 3) modifying the subsequent dose; and wherein the subject is administered the subsequent dose every three weeks.

In some embodiments, the subsequent dose is modified based on the subject's response. In some embodiments, the subsequent dose is modified based on the subject's response, and wherein the subject's response is a change in red blood cell transfusion burden. In some embodiments, the subsequent dose is modified based on the subject's red blood cell transfusion burden after at least two consecutive doses. In some embodiments, the dosing regimen is for the treatment of thalassemia, and the subsequent dose is increased to 1.25 mg/kg. In some embodiments, the subsequent dose is increased to 1.25 mg/kg in a subject with no reduction in red blood cell transfusion burden. In some embodiments, the dosing regimen is for the treatment of myelodysplastic syndrome, and wherein the subsequent dose is increased to 1.33 mg/kg or 1.75 mg/kg. In some embodiments, the subsequent dose is increased to 1.33 mg/kg or 1.75 mg/kg in a subject with no reduction in red blood cell transfusion burden. In some embodiments, the subsequent dose is interrupted or discontinued. In some embodiments, the subsequent dose is discontinued in a subject with no reduction in transfusion burden after three consecutive doses. In some embodiments, the subsequent dose is modified based on the subject's response, and the subject's response is a change the subject's pre-dose hemoglobin levels. In some embodiments, the subject has a pre-dose hemoglobin level greater than or equal to 11.5 g/dL in the absence of red blood cell transfusions. In some embodiments, the subsequent dose is interrupted or discontinued. In some embodiments, the subject's pre-dose hemoglobin levels increase greater than 2 g/dL in the absence of red blood cell transfusions, and wherein the increase occurs within three weeks of administration. In some embodiments, the subsequent dose is reduced. In some embodiments, the subsequent dose is reduced to 1.33 mg/kg, 1.0 mg/kg, 0.8 mg/kg, 0.6 mg/kg, or discontinued. In some embodiments, the subsequent dose is modified if the subject experiences a grade 3 or higher adverse reaction. In some embodiments, the dose is interrupted or discontinued if the subject experiences a grade 3 or higher adverse reaction.

In some embodiments, the dosing regimen is administered to a subject with β-thalassemia. In some embodiments, the dosing regimen is administered to a subject with α-thalassemia. In some embodiments, the subject has very low to intermediate-risk myelodysplastic syndrome with ring sideroblasts (MDS-RS). In some embodiments, the subject has myelodysplastic or myeloproliferative neoplasm with ring sideroblasts. In some embodiments, the subject has thrombocytosis.

In some embodiments, the subject experiences a reduction in red blood cell transfusion burden. In some embodiments, the subject experiences a reduction in red blood cell transfusion burden for at least 12 consecutive weeks. In some embodiments, the subject experiences a 33% or greater reduction in red blood cell transfusion burden relative to the subject's baseline transfusion burden. In some embodiments, the subject experiences a 50% or greater reduction in red blood cell transfusion burden relative to the subject's baseline transfusion burden. In some embodiments, the subject becomes red blood cell transfusion independent. In some embodiments, the subject becomes red blood cell transfusion independent for at least eight consecutive weeks. In some embodiments, the subject becomes red blood cell transfusion independent for at least twelve consecutive weeks.

In some embodiments, the polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 3. In some embodiments, the polypeptide consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 3. In some embodiments, the polypeptide is part of a homodimer protein complex.

In some embodiments, the initial dose or subsequent dose is administered parenterally. In some embodiments, the initial dose or subsequent dose is administered via subcutaneous injection.

In some embodiments, the polypeptide is provided as a lyophilized powder in a vial. In some embodiments, the lyophilized powder is provided in an amount of 25 mg/vial or 75 mg/vial.

In some embodiments, the lyophilized powder is reconstituted with sterile water for injection. In some embodiments, the lyophilized powder is reconstituted with Sterile Water for Injection to a final polypeptide concentration of approximately 45 mg/mL, 46 mg/mL, 47 mg/mL, 48 mg/mL, 49 mg/mL, 50 mg/mL, 51 mg/mL, 52 mg/mL, 53 mg/mL, 54 mg/mL, or 55 mg/mL. In some embodiments, the lyophilized powder is reconstituted with Sterile Water for Injection to a final polypeptide concentration of approximately 50 mg/mL. In some embodiments, the lyophilized powder is reconstituted with approximately 0.5 mL, 0.56 mL, 0.58 mL, 0.6 mL, 0.62 mL, 0.64 mL, 0.66 mL, 0.68 mL, 0.70 mL, 0.72 mL, 0.74 mL, 0.76 mL, 0.78 mL, 0.8 mL, 0.82 mL, 0.84 mL, 0.86 mL, 0.88 mL, 0.9 mL, 0.92 mL, 0.94 mL, 0.96 mL, 0.98 mL, 1 mL, 1.1 mL, 1.15 mL, 1.2 mL, 1.25 mL, 1.3 mL, 1.35 mL, 1.4 mL, 1.45 mL, 1.5 mL, 1.55 mL, 1.6 mL, 1.65 mL, 1.7 mL, 1.75 mL, or 1.8 mL of Sterile Water for Injection. In some embodiments, the lyophilized powder for injection is provided in an amount of 25 mg/vial, and wherein the polypeptide is reconstituted with 0.65 mL, 0.66 mL, 0.67 mL, 0.68 mL, 0.69 mL, 0.70 mL, 0.71 mL, 0.72 mL, 0.73 mL, 0.74 mL, or 0.75 mL of Sterile Water for Injection. In some embodiments, the lyophilized powder for injection is provided in an amount of 75 mg/vial, and wherein the polypeptide is reconstituted with 0.65 mL, 0.66 mL, 1.55 mL, 1.56 mL, 1.57 mL, 1.58 mL, 1.59 mL, 1.6 mL, 1.61 mL, 1.62 mL, 1.63 mL, 1.64 mL, 1.65 mL, 1.66 mL, 1.67 mL, 1.68 mL, 1.69 mL, 1.7 mL, 1.71 mL, 1.72 mL, 1.73 mL, 1.74 mL, or 1.75 mL of Sterile Water for Injection.

In some embodiments, the vial comprises a lyophilized powder and one or more pharmaceutical additives and/or excipients. In some embodiments, one or more of the pharmaceutical additives and/or excipients is a buffering agent. In some embodiments, the buffering agent is selected to be physiologically compatible and to maintain a pH of 5.5, 5.7, 6.0, 6.3, 6.5, 6.7, 7.0, 7.3, 7.5, 7.7, 8.0, 8.3, 8.5, 8.7, 9.0, 9.3, 9.5, 9.7, or 10.0 when reconstituted with Sterile Water for Injection. In some embodiments, the buffering agent is selected to be physiologically compatible and to maintain a pH of 6.0, 6.3, 6.5, 6.7, 7.0, 7.3, or 7.5 when reconstituted with Sterile Water for Injection. In some embodiments, the buffering agent is selected to be physiologically compatible and to maintain a pH of 6.5 when reconstituted with Sterile Water for Injection. In some embodiments, the buffering agent comprises organic acids, succinate, phosphate, acetate, citrate, citric acid. Tris. HEPES, amino acids, or mixtures of amino acids. In some embodiments, the buffering agent comprises tri-sodium citrate dihydrate. In some embodiments, the buffering agent comprises citric acid monohydrate. In some embodiments, the buffering agent comprises tri-sodium citrate dihydrate and citric acid monohydrate. In some embodiments, the buffering agent comprises a concentration of at least 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 25, 30, 40, 50, 60, 70, 80, 90, 100, 200, or 500 mM. In some embodiments, the buffering agent comprises a concentration of at least 10 mM.

In some embodiments, one or more of the pharmaceutical additives and/or excipients is a stabilizer. In some embodiments, the stabilizer is selected from the group consisting of: sucrose, trehalose, mannose, maltose, lactose, glucose, raffinose, cellobiose, gentiobiose, isomaltose, arabinose, glucosamine, fructose, mannitol, sorbitol, poly-hydroxy compounds, polysaccharides, dextran, starch, hydroxyethyl starch, cyclodextrins, N-methyl pyrollidene, cellulose, or hyaluronic acid. In some embodiments, the stabilizer is sucrose. In some embodiments, the stabilizer comprises a concentration of at least 0.005% w/v, 0.01% w/v, 0.02% w/v, 0.03% w/v, 0.05% w/v, 0.06% w/v, 0.07% w/v, 0.08% w/v, 0.09% w/v, 0.1% w/v, 0.5% w/v, 0.7% w/v, 0.8% w/v, 0.9% w/v, 1.0% w/v, 1.2% w/v, 1.5% w/v, 1.7% w/v, 2% w/v, 3% w/v, 4% w/v, 5% w/v, 6% w/v, 7% w/v, 8% w/v, 9% w/v, 10% w/v, 11% w/v, 12% w/v, 13% w/v, 14% w/v, 15% w/v, 16% w/v, 17% w/v, 18% w/v, 19% w/v, or 20% w/v. In some embodiments, the stabilizer comprises a concentration of at least 9% w/v.

In some embodiments, one or more of the pharmaceutical additives and/or excipients is a surfactant. In some embodiments, the surfactant is selected from the group consisting of: sodium lauryl sulfate, dioctyl sodium sulfosuccinate and dioctyl sodium sulfonate, chenodeoxycholic acid, N-lauroylsarcosine sodium salt, lithium dodecyl sulfate, 1-octanesulfonic acid sodium salt, sodium cholate hydrate, sodium deoxycholate, and glycodeoxycholic acid sodium salt, benzalkonium chloride, benzethonium chloride, cetylpyridinium chloride monohydrate, hexadecyltrimethylammonium bromide, CHAPS, CHAPSO, SB3-10, SB3-12, digitonin, Triton X-100, Triton X-114, TWEEN-20, TWEEN-80, lauromacrogol 400, polyoxyl 40 stearate, polyoxyethylene hydrogenated castor oil 10, 40, 50 and 60, glycerol monostearate, polysorbate 40, polysorbate 60, polysorbate 65, polysorbate 80, or soy lecithin. In some embodiments, the surfactant is polysorbate 80. In some embodiments, the surfactant comprises a concentration of at least 0.001, 0.002, 0.003, 0.004, 0.005, 0.01, 0.02, 0.03, 0.05, 0.06, 0.07, 0.08, 0.09, 0.1, 0.5, 0.7, 0.8 0.9, or 1.0% w/v. In some embodiments, the surfactant comprises a concentration of at least 0.2% w/v.

In some embodiments, the vial comprises a lyophilized powder comprising the polypeptide, citric acid monohydrate, tri-sodium citrate dehydrate, polysorbate 80, and sucrose. In some embodiments, the vial comprises a lyophilized powder comprising 37.5 mg of the polypeptide, 0.127 mg citric acid monohydrate, 2.029 mg tri-sodium citrate dehydrate, 0.15 mg polysorbate 80, and 67.5 mg sucrose. In some embodiments, the vial comprises a lyophilized powder comprising 87.5 mg ActRII polypeptide, 0.296 mg citric acid monohydrate, 4.734 mg tri-sodium citrate dehydrate, 0.35 mg polysorbate 80, and 157.5 mg sucrose.

### BRIEF DESCRIPTION OF THE DRAWINGS

FIG. 1 shows components of a kit comprising a lyophilized polypeptide and an injection device. A vial (1) holds lyophilized polypeptide, reconstituted sterile injectable solution, or sterile injectable solution. A prefilled syringe (2) containing a reconstitution solution is used to reconstitute lyophilized polypeptide from (1) into a sterile injectable solution. A vial adapter (3) couples the vial (1) to the pre-filled syringe (2) via attachment to the vial at one end, and attachment to the pre-filled syringe at an opposite end. A syringe (4) and needle (5) are provided for administration of sterile injectable solution. Swab wipes (6) are provided for sterilization of individual kit components.

### DETAILED DESCRIPTION

1. Overview

Provided herein are dosing regimens, dosage forms, and formulations comprising a recombinant fusion protein comprising an extracellular domain (ECD) of human activin receptor type-II (ActRII) polypeptides or derivatives thereof linked to a constant domain of an immunoglobulin, such as human IgG1 Fc domain. In certain aspects, the disclosure relates to a lyophilized powder comprising an extracellular domain (ECD) of a human ActRII polypeptide or derivatives thereof linked to a constant domain of an immunoglobulin, such as human IgG1 Fe domain for reconstitution into a sterile solution for injection.

2. Dosing Regimens. Dosage Forms, and Formulations

In certain aspects, the disclosure relates to dosing regimens, dosage forms, and formulations comprising a lyophilized powder comprising an extracellular domain (ECD) of human ActRII polypeptide or derivatives thereof linked to a constant domain of an immunoglobulin, such as human IgG1 Fc domain for reconstitution into a sterile solution for injection. During lyophilization, the polypeptide is converted from being in an aqueous phase to being in an amorphous solid phase, which is thought to protect the protein from chemical and/or conformational instability. Lyophilization is carried out using techniques common in the art and the lyophilized formulations are optimized for stability, shelf-life, and decreased levels of high molecular weight (HMW) species and aggregates. Tang et al., Pharm Res. 21:191-200, (2004) and Chang et al., Pharm Res. 13:243-9 (1996). The dosing regimens, dosage forms, and formulations provided aid in stabilizing the protein against the stresses of manufacturing, shipping and storage. The excipients and additives used in the lyophilized formulations are integral components of a formulation, and therefore need to be safe and well tolerated by patients. For protein drugs, the choice of excipients and additives is particularly important because they can affect both efficacy and immunogenicity of the drug. Excipients and additives are also useful in reducing viscosity of high concentration polypeptide formulations in order to enable their delivery and enhance patient convenience. The formulation excipients and additives disclosed herein provide stability against these stresses. Common excipients are known in the art and can be found in Powell et al., Compendium of Excipients fir Parenteral Formulations (1998), PDA J. Pharm. Sci. Technology, 52:238-311.

In certain aspects, the disclosure provides a dosing regimen for the treatment of thalassemia in a subject in need thereof comprising administering a lyophilized human ActRII polypeptide linked to a constant domain of an immunoglobulin, wherein the dosing regimen comprises: 1) administering an initial dose of 1 mg/kg: 2) monitoring a subject's response; and 3) modifying the subsequent dose; and wherein the subject is administered the subsequent dose every three weeks. In certain aspects, the disclosure provides a dosing regimen for the treatment of myelodysplastic syndrome in a subject in need thereof comprising administering a lyophilized human ActRII polypeptide linked to a constant domain of an immunoglobulin, wherein the dosing regimen comprises: 1) administering an initial dose of 1 mg/kg: 2) monitoring a subject's response; and 3) modifying the subsequent dose; and wherein the subject is administered the subsequent dose every three weeks. In some embodiments, the subsequent dose is modified based on the subject's response.

A subject's red blood cell transfusion dependence or transfusion burden may be monitored in order to modify the subsequent dose administered in the dosing regimen as required. In some embodiments, the subsequent dose is modified based on the subject's response, wherein the subject's response is a change in red blood cell transfusion burden. In some embodiments, the subsequent dose is modified based on the subject's red blood cell transfusion burden after at least two consecutive doses. Subjects with thalassemia may require an increased dose of 1.25 mg/kg based on their response to the dosing regimen. In some embodiments, the dosing regimen is for the treatment of thalassemia, and the subsequent dose is increased to 1.25 mg/kg. In some embodiments, the subsequent dose is increased to 1.25 mg/kg in a subject with no reduction in red blood cell transfusion burden. Subjects with myelodysplastic syndromes may require an increased dose of 1.33 mg/kg or 1.75 mg/kg based on their response to the dosing regimen. In some embodiments, the dosing regimen is for the treatment of myelodysplastic syndrome, and the subsequent dose is increased to 1.33 mg/kg or 1.75 mg/kg. In some embodiments, the subsequent dose is increased to 1.33 mg/kg or 1.75 mg/kg in a subject with no reduction in red blood cell transfusion burden. In some embodiments, the subsequent dose is interrupted or discontinued. In some embodiments, the subsequent dose is discontinued in a subject with no reduction in transfusion burden after three consecutive doses.

A subject's pre-dose hemoglobin levels may be monitored in order to modify the subsequent dose administered in the dosing regimen as required. In some embodiments, the subsequent dose is modified based on the subject's response, and the subject's response is a change the subject's pre-dose hemoglobin levels. In some embodiments, the subject has a pre-dose hemoglobin level greater than or equal to 11.5 g/dL in the absence of red blood cell transfusions. In some embodiments, the subsequent dose is interrupted or discontinued. In some embodiments, the subject's pre-dose hemoglobin levels increase greater than 2 g/dL in the absence of red blood cell transfusions, and wherein the increase occurs within three weeks of administration. In some embodiments, the subsequent dose is reduced. In some embodiments, the subsequent dose is reduced to 1.33 mg/kg, 1.0 mg/kg, 0.8 mg/kg, 0.6 mg/kg, or discontinued.

A dosing regimen disclosed herein may be discontinued in a subject experiencing a grade 3 or higher adverse reaction. Grade refers to the severity of the adverse reaction. Grades 1-4 adverse reactions comprise mild, moderate, severe, and life-threatening or disabling adverse reactions, respectively. In some embodiments, the subsequent dose is modified if the subject experiences a grade 3 or higher adverse reaction. In some embodiments, the dose is interrupted or discontinued if the subject experiences a grade 3 or higher adverse reaction.

In some embodiments, the dosing regimen is administered to a subject with p-thalassemia. In some embodiments, the dosing regimen is administered to a subject with α-thalassemia. In some embodiments, the subject has very low to intermediate-risk myelodysplastic syndrome with ring sideroblasts (MDS-RS). In some embodiments, the subject has myelodysplastic or myeloproliferative neoplasm with ring sideroblasts. In some embodiments, the subject has thrombocytosis. In some embodiments, the subject experiences a reduction in red blood cell transfusion burden. In some embodiments, the subject experiences a reduction in red blood cell transfusion burden for at least 12 consecutive weeks. In some embodiments, the subject experiences a 33% or greater reduction in red blood cell transfusion burden relative to the subject's baseline transfusion burden. In some embodiments, the subject experiences a 50% or greater reduction in red blood cell transfusion burden relative to the subject's baseline transfusion burden. In some embodiments, the subject becomes red blood cell transfusion independent. In some embodiments, the subject becomes red blood cell transfusion independent for at least eight consecutive weeks. In some embodiments, the subject becomes red blood cell transfusion independent for at least twelve consecutive weeks.

In another aspect, provide herein are dosing regimens, lyophilized dosage forms, and formulations comprising lyophilized ActRII polypeptides. In certain embodiments, the disclosure provides a dosing regimen comprising an ActRII polypeptide wherein the polypeptide is in a lyophilized form or in a liquid solution in a vial. In certain embodiments, the dosing regimen comprises about 15 mg, about 17.5 mg, about 20 mg, about 22.5 mg, about 25 mg, about 27.5 mg, about 30 mg, about 32.5 mg, about 35 mg, about 37.5 mg, about 40 mg, about 42.5 mg, about 45 mg, about 47.5 mg, about 50 mg, about 52.5 mg, about 55 mg, about 57.5 mg, about 60 mg, about 62.5 mg, about 65 mg, about 67.5 mg, about 70 mg, about 72.5 mg, about 75 mg, about 77.5 mg, about 80 mg, about 82.5 mg, about 85 mg, about 90 mg, about 92.5 mg, about 95 mg, about 97.5 mg or about 100 mg of the ActRII polypeptide.

In certain embodiments, the ActRII polypeptide is provided as a lyophilized powder for solution for injection in vials. In specific embodiments, the ActRII polypeptide for injection is provided in an amount of 25 mg/vial or 75 mg/vial. In more specific embodiments, each of said 25 mg/vial or said 75 mg/vial is reconstituted with Sterile Water for Injection, each containing a final concentration of 45 to 55 mg/mL of the reconstituted ActRII polypeptide. In a more specific embodiment, each of said 25 mg/vial or said 75 mg/vial is reconstituted with Sterile Water for Injection, each containing a final concentration of 50 mg/mL of the reconstituted ActRII polypeptide (active pharmaceutical ingredient).

In certain embodiments, the ActRII polypeptide for injection is provided in an amount of 25 mg/vial or 75 mg/vial, and is reconstituted with Sterile Water for Injection. In a more specific embodiment, the ActRII polypeptide for injection provided in each of said 25 mg/vial or said 75 mg/vial is reconstituted with Sterile Water for Injection to a final concentration of approximately 45 mg/mL, 46 mg/mL, 47 mg/mL, 48 mg/mL, 49 mg/mL, 50 mg/mL, 51 mg/mL, 52 mg/mL, 53 mg/mL, 54 mg/mL, or 55 mg/mL of the reconstituted ActRII polypeptide. n a more specific embodiment, the ActRII polypeptide for injection provided in each of said 25 mg/vial or said 75 mg/vial is reconstituted with Sterile Water for Injection to a final concentration of 45 mg/mL, 46 mg/mL, 47 mg/mL, 48 mg/mL, 49 mg/mL, 50 mg/mL, 51 mg/mL, 52 mg/mL, 53 mg/mL, 54 mg/mL, or 55 mg/mL of the reconstituted ActRII polypeptide. In some embodiments, the ActRII polypeptide for injection provided in each of said 25 mg/vial or said 75 mg/vial is reconstituted with Sterile Water for Injection to a final concentration of approximately 50 mg/mL of the reconstituted ActRII polypeptide. In some embodiments, the ActRII polypeptide for injection provided in each of said 25 mg/vial or said 75 mg/vial is reconstituted with Sterile Water for Injection to a final concentration of 50 mg/mL of the reconstituted ActRII polypeptide.

In some embodiments, the ActRII polypeptide for injection provided in each of said 25 mg/vial or said 75 mg/vial is reconstituted with 0.5 to 2 mL of Sterile Water for Injection. In some embodiments, the ActRII polypeptide for injection provided in each of said 25 mg/vial or said 75 mg/vial is reconstituted with approximately 0.5 mL, 0.56 mL, 0.58 mL, 0.6 mL, 0.62 mL, 0.64 mL, 0.66 mL, 0.68 mL, 0.70 mL, 0.72 mL, 0.74 mL, 0.76 mL, 0.78 mL, 0.8 mL, 0.82 mL, 0.84 mL, 0.86 mL, 0.88 mL, 0.9 mL, 0.92 mL, 0.94 mL, 0.96 mL, 0.98 mL, 1 mL, 1.1 mL, 1.15 mL, 1.2 mL, 1.25 mL, 1.3 mL, 1.35 mL, 1.4 mL, 1.45 mL, 1.5 mL, 1.55 mL, 1.6 mL, 1.65 mL, 1.7 mL, 1.75 mL, or 1.8 mL of Sterile Water for Injection. In some embodiments, the ActRII polypeptide for injection provided in each of said 25 mg/vial or said 75 mg/vial is reconstituted with 0.5 mL, 0.55 mL, 0.56 mL, 0.57 mL, 0.58 mL, 0.59 mL, 0.6 mL, 0.65 mL, 0.66 mL, 0.67 mL, 0.68 mL, 0.69 mL, 0.70 mL, 0.71 mL, 0.72 mL, 0.73 mL, 0.74 mL, 0.75 mL, 0.76 mL, 0.77 mL, 0.78 mL, 0.79 mL, 0.8 mL, 0.85 mL, 0.9 mL, 0.95 mL, 1 mL, 1.1 mL, 1.15 mL, 1.2 mL, 1.25 mL, 1.3 mL, 1.35 mL, 1.4 mL, 1.45 mL, 1.5 mL, 1.51 mL, 1.52 mL, 1.53 mL, 1.54 mL, 1.55 mL, 1.56 mL, 1.57 mL, 1.58 mL, 1.59 mL, 1.6 mL, 1.61 mL, 1.62 mL, 1.63 mL, 1.64 mL, 1.65 mL, 1.66 mL, 1.67 mL, 1.68 mL, 1.69 mL, 1.7 mL, 1.71 mL, 1.72 mL, 1.73 mL, 1.74 mL, 1.75 mL, 1.76 mL, 1.77 mL, 1.78 mL, 1.79 mL, or 1.8 mL of Sterile Water for Injection. In some embodiments, the ActRII polypeptide for injection provided in said 25 mg/vial is reconstituted with 0.65 mL, 0.66 mL, 0.67 mL, 0.68 mL, 0.69 mL, 0.70 mL, 0.71 mL, 0.72 mL, 0.73 mL, 0.74 mL, or 0.75 mL of Sterile Water for Injection. In some embodiments, the ActRII polypeptide for injection provided in said 25 mg/vial is reconstituted with 1.55 mL, 1.56 mL, 1.57 mL, 1.58 mL, 1.59 mL, 1.6 mL, 1.61 mL, 1.62 mL, 1.63 mL, 1.64 mL, 1.65 mL, 1.66 mL, 1.67 mL, 1.68 mL, 1.69 mL, 1.7 mL, 1.71 mL, 1.72 mL, 1.73 mL, 1.74 mL, or 1.75 mL of Sterile Water for Injection.

In some embodiments, the vials of ActRII polypeptide provided herein comprise a lyophilized powder ActRII polypeptide and one or more pharmaceutical additives and/or excipients. In certain embodiments the one or more pharmaceutical additives and/or excipients comprises a buffer, a bulking agent, stabilizer, and/or a surfactant. In certain embodiments, the one or more pharmaceutical additives and/or excipients comprises a surfactant, buffering agent, stabilizer, and/or anticoagulant. Buffering agents may be selected to maintain the pH of the formulation during processing and upon reconstitution. Stabilizers may include cryo and lyoprotectants, such as polyols, sugars, and polysaccharides, and may be selected to protect the formulation from freeze/thaw cycle stresses and stabilize the formulation in the freeze-dried state. Surfactants may be selected based on their ability to serve as an emulsifier, wetter, solubilizer and/or dispersant.

The dosing regimen and formulations provided herein comprise buffering agents, stabilizing agents, surfactants, sugars, salts and/or amino acids, which are described in greater detail below.

A person having ordinary skill in the art would recognize that the concentrations of the excipients described herein share an interdependency within a particular formulation. By way of example, the concentration of a bulking agent is, in one aspect, lowered where, e.g., there is a high protein concentration or where, e.g., there is a high stabilizing agent concentration. In addition, a person having ordinary skill in the art would recognize that, in order to maintain the isotonicity of a particular formulation in which there is no bulking agent, the concentration of a stabilizing agent could be increased accordingly (i.e., a “tonicifying” amount of stabilizer would be used. Excipients and other additives are added to impart or enhance manufacturability and/or final product quality, such as the stability and delivery of a drug product (e.g., protein). The dosing regimen and formulations provided herein comprise suitable excipients that enhance stability, and safety.

(a) Buffering Agent

Typically, the stability of a pharmacologically active polypeptide formulation is observed to be maximal in a narrow pH range. This pH range of optimal stability needs to be identified early during pre-formulation studies. Several approaches, such as accelerated stability studies and calorimetric screening studies, are useful in this endeavor (Remmele R. L. Jr., et al., Biochemistry, 38(16): 5241-7 (1999)). Once a formulation is finalized, the protein must be manufactured and maintained throughout its shelf-life. Hence, buffering agents are almost always employed to control pH in the formulation.

Several factors must be considered when choosing a buffering agent. First and foremost, the buffer species and its concentration must be defined based on its pKa and the desired formulation pH. Equally important is to ensure that the buffer is compatible with the protein and other formulation excipients, and does not catalyze any degradation reactions. A third important aspect to be considered is the sensation of stinging and irritation the buffer may induce upon administration. The potential for stinging and irritation is greater for drugs that are administered via the subcutaneous (SC) or intramuscular (IM) routes, where the drug solution remains at the site for a relatively longer period of time than when administered by the IV route where the formulation gets diluted rapidly into the blood upon administration. For formulations that are administered by direct IV infusion, the total amount of buffer (and any other formulation component) needs to be monitored.

Buffers for lyophilized formulations require additional consideration. For example, particular buffers such as sodium phosphate have a propensity to crystallize out of the protein amorphous phase during freezing resulting in shifts in pH. In certain embodiments, exemplary buffering agents used to buffer the dosing regimens, dosage forms, and formulations as set forth herein include, but are not limited to organic acids, succinate, phosphate, acetate, citrate, Tris, HEPES, and amino acids or mixtures of amino acids, including, but not limited to aspartate, histidine, arginine and glycine. In some embodiments, the buffering agent comprises tri-sodium citrate dihydrate. In some embodiments, the buffering agent comprises citric acid monohydrate. In a preferred embodiment, the buffering agent comprises tri-sodium citrate dihydrate and citric acid monohydrate. In a preferred embodiment, the buffering agents are tri-sodium citrate dihydrate and citric acid monohydrate.

In one embodiment, the buffering agent present in the formulation is selected to be physiologically compatible and to maintain a desired pH of the pharmaceutical formulation when reconstituted with Sterile Water for Injection. In another embodiment, the pH of the solution is between pH 2.0 and pH 12.0. For example, in various embodiments the pH of the reconstituted solution may be 5.5, 5.7, 6.0, 6.3, 6.5, 6.7, 7.0, 7.3, 7.5, 7.7, 8.0, 8.3, 8.5, 8.7, 9.0, 9.3, 9.5, 9.7, or 10.0. In some embodiments, the buffering agent maintains a pH range from pH 6-7.5 when reconstituted in solution. In some embodiments, the pH of the reconstituted solution may be 6.0, 6.3, 6.5, 6.7, 7.0, 7.3, or 7.5. In a preferred embodiment, the pH of the reconstituted solution may be 6.5.

The pH buffering compound may be present in any amount suitable to maintain the pH of the formulation at a predetermined level. When appropriately low levels of buffer are used, crystallization and pH shifts may be avoided. In one embodiment, the concentration of the buffering agent is between 0.1 mM and 500 mM (1 M). For example, it is contemplated that the buffering agent is at least 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 25, 30, 40, 50, 60, 70, 80, 90, 100, 200, or 500 mM.

(b) Stabilizers

In certain embodiments the dosing regimens, dosage forms, and formulations provided herein comprise stabilizers. These stabilizers can be classified on the basis of the mechanisms by which they stabilize proteins against various chemical and physical stresses. Some stabilizers are used to alleviate the effects of a specific stress or to regulate a particular susceptibility of a specific protein. Other stabilizers have more general effects on the physical and covalent stabilities of proteins. Given the teachings and guidance provided herein, those skilled in the art will know what amount or range of stabilizer can be included in any particular formulation to achieve a formulation of the disclosure that is likely to promote retention and stability of the ActRII polypeptide.

In some embodiments, a stabilizer (or a combination of stabilizers) may be added to the formulation to prevent or reduce storage-induced aggregation and chemical degradation. A hazy or turbid solution upon reconstitution normally indicates that the protein has precipitated or at least aggregated. Stabilizers are capable of preventing aggregation, or chemical degradation (for example, autolysis, deamidation, oxidation, etc.). Some stabilizers are also capable of acting as anticoagulants upon administration of the formulation to a patient. In certain embodiments, the dosing regimens, dosage forms, and formulations provided herein include stabilizers including but not limited to, sucrose, trehalose, mannose, maltose, lactose, glucose, raffinose, cellobiose, gentiobiose, isomaltose, arabinose, glucosamine, fructose, mannitol, sorbitol, poly-hydroxy compounds, including polysaccharides such as dextran, starch, hydroxyethyl starch, cyclodextrins, N-methyl pyrollidene, cellulose and hyaluronic acid [Carpenter et al., Develop. Biol. Standard 74:225, (1991)]. In one embodiment of the disclosure, sucrose is used as a stabilizer.

In certain embodiments the formulation comprises a stabilizer in a concentration of about 0.11, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, 90, 100, 200, 500, 700, 900, or 1000 mM. Likewise, in certain embodiments of the disclosure, the stabilizer is incorporated in a concentration of about 0.005, 0.01, 0.02, 0.03, 0.05, 0.06, 0.07, 0.08, 0.09, 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, or 20% w/v.

In other embodiments, the dosing regimens, dosage forms, and formulations provided herein may include appropriate amounts of bulking and osmolarity regulating agents. These bulking and osmolarity regulating agents may include, for example, polymers such as dextran, polyvinylpyrolidone, carboxymethylcellulose, lactose, sorbitol, trehalose, or xylitol.

(c) Surfactants

In certain embodiments, the dosing regimens, dosage forms, and formulations provided herein may additionally include surfactants. Surfactants are commonly used in protein formulations to prevent surface-induced degradation. Surfactants are amphipathic molecules with the capability of out-competing proteins for interfacial positions (and/or promote proper refolding of a structurally altered protein molecule). Hydrophobic portions of the surfactant molecules occupy interfacial positions (e.g., air/liquid), while hydrophilic portions of the molecules remain oriented towards the bulk solvent. At sufficient concentrations (typically around the detergent's critical micellar concentration), a surface layer of surfactant molecules serve to prevent protein molecules from adsorbing at the interface. Thereby, surface-induced degradation is minimized. Surfactants contemplated herein include, without limitation, fatty acid esters of sorbitan polyethoxylates, i.e. polysorbate 20 and polysorbate 80. The two differ only in the length of the aliphatic chain that imparts hydrophobic character to the molecules, C-12 and C-18, respectively. Accordingly, polysorbate-80 is more surface-active and has a lower critical micellar concentration than polysorbate-20.

Detergents can also affect the thermodynamic conformational stability of proteins. Non-ionic surfactants are generally useful in protein stabilization. Ionic surfactants (detergents) normally destabilize proteins. Here again, the effects of a given detergent excipient will be protein specific. For example, polysorbates have been shown to reduce the stability of some proteins and increase the stability of others. Detergent destabilization of proteins can be rationalized in terms of the hydrophobic tails of the detergent molecules that can engage in specific binding with partially or wholly unfolded protein states. These types of interactions could cause a shift in the conformational equilibrium towards the more expanded protein states (i.e. increasing the exposure of hydrophobic portions of the protein molecule in complement to binding polysorbate). Alternatively, if the protein native state exhibits some hydrophobic surfaces, detergent binding to the native state may stabilize that conformation. Another aspect of polysorbates is that they are inherently susceptible to oxidative degradation. Often, as raw materials, they contain sufficient quantities of peroxides to cause oxidation of protein residue side-chains, especially methionine. The potential for oxidative damage arising from the addition of stabilizer emphasizes the point that the lowest effective concentrations of excipients should be used in formulations. For surfactants, the effective concentration for a given protein will depend on the mechanism of stabilization.

Surfactants are also added in appropriate amounts to prevent surface related aggregation phenomenon during freezing and drying [Chang, B. J. Pharm. Sci. 85:1325, (1996)]. Thus, exemplary surfactants include, without limitation, anionic, cationic, nonionic, zwitterionic, and amphoteric surfactants including surfactants derived from naturally-occurring amino acids. Anionic surfactants include, but are not limited to, sodium lauryl sulfate, dioctyl sodium sulfosuccinate and dioctyl sodium sulfonate, chenodeoxycholic acid, N-lauroylsarcosine sodium salt, lithium dodecyl sulfate, 1-octanesulfonic acid sodium salt, sodium cholate hydrate, sodium deoxycholate, and glycodeoxycholic acid sodium salt. Cationic surfactants include, but are not limited to, benzalkonium chloride or benzethonium chloride, cetylpyridinium chloride monohydrate, and hexadecyltrimethylammonium bromide. Zwitterionic surfactants include, but are not limited to, CHAPS, CHAPSO, SB3-10, and SB3-12. Non-ionic surfactants include, but are not limited to, digitonin, Triton X-100, Triton X-114. TWEEN-20, and TWEEN-80. Surfactants also include, but are not limited to lauromacrogol 400, polyoxyl 40 stearate, polyoxyethylene hydrogenated castor oil 10, 40, 50 and 60, glycerol monostearate, polysorbate 40, polysorbate 60, polysorbate 65 and polysorbate 80, soy lecithin and other phospholipids such as dioleyl phosphatidyl choline (DOPC), dimyristoylphosphatidyl glycerol (DMPG), dimyristoylphosphatidyl choline (DMPC), and (dioleyl phosphatidyl glycerol) DOPG; sucrose fatty acid ester, methyl cellulose and carboxymethyl cellulose. Formulations comprising these surfactants, either individually or as a mixture in different ratios, are therefore further provided. In one embodiment of the present disclosure, the surfactant is polysorbate 80. In the present formulations, the surfactant is incorporated in a concentration of about 0.01 to about 0.5 g/L. In various embodiments of the dosing regimens, dosage forms, and formulations provided herein, the surfactant concentration is 0.005, 0.01, 0.02, 0.03, 0.05, 0.06, 0.07, 0.08, 0.09, 0.1, 0.2, 0.3, 0.4, 0.5, 0.6, 0.7, 0.8, 0.9 or 1.0 g/L. Likewise, in certain embodiments of the disclosure, the surfactant is incorporated in a concentration of about 0.001, 0.002, 0.003, 0.004, 0.005, 0.01, 0.02, 0.03, 0.05, 0.06, 0.07, 0.08, 0.09, 0.1, 0.5, 0.7, 0.8 0.9, or 1.0% w/v.

(d) Other Pharmaceutical Additives and/or Excipients

In certain embodiments, the dosing regimens, dosage forms, and formulations provided herein may include salts, amino acids, antioxidants, metal ions, and/or preservatives.

Salts are often added to increase the ionic strength of the formulation, which can be important for protein solubility, physical stability, and isotonicity. Salts can affect the physical stability of proteins in a variety of ways. Ions can stabilize the native state of proteins by binding to charged residues on the protein's surface. Alternatively, salts can stabilize the denatured state by binding to peptide groups along the protein backbone (—CONH—). Salts can also stabilize the protein native conformation by shielding repulsive electrostatic interactions between residues within a protein molecule. Salts in protein formulations can also shield attractive electrostatic interactions between protein molecules that can lead to protein aggregation and insolubility. Salts (i.e., electrolytes) sometimes make it more difficult to freeze dry the formulation. For this reason, only sufficient salt to maintain protein structural stability should be included in the formulation, and normally this level of electrolyte is very low. In certain embodiments, the dosing regimens, dosage forms, and formulations provided herein may include salts such as for example sodium chloride (NaCl), Calcium chloride (CaCl₂). Zinc chloride (ZnCl₂), and/or Magnesium chloride (MgCl₂) salts. In certain embodiments, the dosing regimens, dosage forms, and formulations provided herein have a salt concentration of the formulations is between 0.0 (i.e., no salt), 0.001, 0.002, 0.003, 0.004, 0.005, 0.006, 0.007, 0.008, 0.009, 0.010, 0.011, 0.012, 0.013, 0.014, 0.015, 0.020, 0.050, 0.080, 0.1, 1, 10, 20, 30, 40, 50, 80, 100, 120, 150, 200, 300, and 500 mM.

Amino acids have found versatile use in protein formulations as buffering agents, bulking agents, stabilizers, and antioxidants. Thus, some embodiments of the dosing regimens, dosage forms, and formulations provided herein include amino acids such as for example glycine, arginine, histidine, alanine, proline, serine, and glutamic acid. These amino acids often provide multiple benefits to the polypeptide formulations. Histidine is commonly found in marketed protein formulations, and this amino acid provides an alternative to citrate, a buffer known to sting upon injection. Interestingly, histidine has also been reported to have a stabilizing effect, with respect to aggregation when used at high concentrations in both liquid and lyophilized presentations (Chen B. et al., Pharm Res., 20(12): 1952-60 (2003)). Histidine was also observed by others to reduce the viscosity of a high protein concentration formulation. In other aspects, formulations are provided which include one or more of the amino acids glycine, arginine and alanine, and have been shown to stabilize proteins by the mechanism of preferential exclusion. Glycine is also a commonly used bulking agent in lyophilized formulations. Arginine has been shown to be an effective agent in inhibiting aggregation and has been used in both liquid and lyophilized formulations. In the dosing regimens, dosage forms, and formulations provided, the amino acid concentration is between 0.1, 1, 10, 20, 30, 40, 50, 80, 100, 120, 150, 200, 300, and 500 mM.

Oxidation of protein residues arises from a number of different sources. Beyond the addition of specific antioxidants, the prevention of oxidative protein damage involves the careful control of a number of factors throughout the manufacturing process and storage of the product such as atmospheric oxygen, temperature, light exposure, and chemical contamination. The disclosure therefore contemplates the use of the pharmaceutical antioxidants including, without limitation, reducing agents, oxygen/free-radical scavengers, or chelating agents. Antioxidants in therapeutic protein formulations are, in one aspect, water-soluble and remain active throughout the product shelf-life. Reducing agents and oxygen/free-radical scavengers work by ablating active oxygen species in solution. In some embodiments of the dosing regimens, dosage forms, and formulations provided herein, the antioxidant concentration is 0.005, 0.01, 0.02, 0.03, 0.05, 0.06, 0.07, 0.08, 0.09, 0.1, 0.2, 0.3, 0.4, 0.5, 0.6, 0.7, 0.8, 0.9 or 1.0 mg/mL.

In certain embodiments, the dosing regimens, dosage forms, and formulations provided herein may include metal ions. In general, transition metal ions are undesired in protein formulations because they can catalyze physical and chemical degradation reactions in proteins. However, specific metal ions are included in formulations when they are cofactors to proteins and in suspension formulations of proteins where they form coordination complexes (e.g., zinc suspension of insulin). Recently, the use of magnesium ions (10-120 mM) has been proposed to inhibit the isomerization of aspartic acid to isoaspartic acid (WO 2004039337).

In certain embodiments, the dosing regimens, dosage forms, and formulations provided herein may include one or more preservatives. Preservatives are necessary when developing multi-use parenteral formulations that involve more than one extraction from the same container. Their primary function is to inhibit microbial growth and ensure product sterility throughout the shelf-life or term of use of the drug product. Commonly used preservatives include, without limitation, benzyl alcohol, phenol and m-cresol. Although preservatives have a long history of use, the development of protein formulations that includes preservatives can be challenging. Preservatives almost always have a destabilizing effect (aggregation) on proteins, and this has become a major factor in limiting their use in multi-dose protein formulations (Roy S, et al., J Pharm Sci., 94(2): 382-96 (2005)).

(e) Preferred ActRII Polypeptide Formulations

In some embodiments of the dosing regimens, dosage forms, and formulations provided herein, the vials of ActRII polypeptide provided herein comprise a lyophilized powder ActRII polypeptide and one or more pharmaceutical additives and/or excipients. In a preferred embodiment of the dosing regimens, dosage forms, and formulations provided herein, a vial of ActRII polypeptide comprises a lyophilized powder comprising 37.5 mg of ActRII polypeptide: 0.127 mg citric acid monohydrate, 2.029 mg tri-sodium citrate dehydrate, 0.15 mg polysorbate 80, and 67.5 mg sucrose. In a more specific embodiment, said vial is rehydrated with 0.68 mL liquid, e.g., sterile water for injection. In another preferred embodiment of the dosing regimens, dosage forms, and formulations provided herein, a vial of ActRII polypeptide comprises a lyophilized powder comprising 87.5 mg ActRII polypeptide: 0.296 mg citric acid monohydrate, 4.734 mg tri-sodium citrate dehydrate, 0.35 mg polysorbate 80, and 157.5 mg sucrose. In a more specific embodiment, said vial is rehydrated with 1.6 mL liquid, e.g., sterile water for injection. In a more specific embodiment of either of the vials, the vial may additionally comprise NaOH or HCl sufficient to adjust pH. In more specific embodiments, said vials comprise one, two, or all three of citrate, e.g., 10 mM citrate; sucrose, e.g., 9% (w/v) sucrose; and/or polysorbate 80, e.g., at pH 6.0 to 7.0, e.g., 0.02% (w/v) polysorbate 80 at pH 6.5.

In certain embodiments the dose is administered parenterally. In some embodiments, the dose is administered via subcutaneous injection. In some embodiments, the dose is administered via intradermal injection. In some embodiments, the dose is administered via intramuscular injection. In some embodiments, the dose is administered via intravenous injection. In some embodiments, the dose is self-administered.

3. Other Pharmaceutical Compositions & Modes of Administration

In certain embodiments, the methods of the disclosure include administering the formulation systemically, or locally. When administered, the therapeutic composition for use in this disclosure is in a substantially pyrogen-free, or pyrogen-free, physiologically acceptable form. Therapeutically useful agents other than the ActRII polypeptides which may also optionally be included in the composition as described above, may be administered simultaneously or sequentially with the subject compounds in the methods disclosed herein.

Typically, protein therapeutic agents disclosed herein will be administered parentally, and particularly intravenously or subcutaneously. Pharmaceutical compositions suitable for parenteral administration may comprise one or more ActRII polypeptides in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. The formulations can be presented in unit-dose or multi-dose sealed containers, such as ampules and vials, and can be stored in a freeze-dried (lyophilized) condition requiring only the addition of a sterile liquid excipient, for example, water, for injections, immediately prior to use. Extemporaneous injection solutions and suspensions can be prepared from sterile powders, granules, and tablets of the kind described herein.

The compositions and formulations may, if desired, be presented in a vial, container, pack, or dispenser device which may contain one or more unit dosage forms containing the active ingredient. The vial, container, pack, or dispenser device may for example comprise metal or plastic foil, such as a blister pack. The vial, container, pack, or dispenser device or dispenser device may be accompanied by instructions for administration.

Further, the composition may be encapsulated or injected in a form for delivery to a target tissue site. In certain embodiments, compositions of the present disclosure may include a matrix capable of delivering one or more therapeutic compounds (e.g., ActRII polypeptides) to a target tissue site, providing a structure for the developing tissue and optimally capable of being resorbed into the body. For example, the matrix may provide slow release of the ActRII polypeptide. Such matrices may be formed of materials presently in use for other implanted medical applications.

The choice of matrix material is based on biocompatibility, biodegradability, mechanical properties, cosmetic appearance and interface properties. The particular application of the subject compositions will define the appropriate formulation. Potential matrices for the compositions may be biodegradable and chemically defined calcium sulfate, tricalcium phosphate, hydroxyapatite, polylactic acid and polyanhydrides. Other potential materials are biodegradable and biologically well defined, such as bone or dermal collagen. Further matrices are comprised of pure proteins or extracellular matrix components. Other potential matrices are non-biodegradable and chemically defined, such as sintered hydroxyapatite, bioglass, aluminates, or other ceramics. Matrices may be comprised of combinations of any of the above mentioned types of material, such as polylactic acid and hydroxyapatite or collagen and tricalcium phosphate. The bioceramics may be altered in composition, such as in calcium-aluminate-phosphate and processing to alter pore size, particle size, particle shape, and biodegradability.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

The compositions of the disclosure may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

It is understood that the dosage regimen may be altered by the attending physician considering various factors which modify the action of the subject compounds of the disclosure (e.g., ActRII polypeptides). The various factors include, but are not limited to, the patient's age, sex, and diet, the severity disease, time of administration, and other clinical factors. Optionally, the dosage may vary with the type of matrix used in the reconstitution and the types of compounds in the composition. In some embodiments, ActRII polypeptides of the disclosure are administered at 0.1 mg/kg. In some embodiments, ActRII polypeptides of the disclosure are administered at 0.2 mg/kg. In some embodiments, ActRII polypeptides of the disclosure are administered at 0.3 mg/kg. In some embodiments. ActRII polypeptides of the disclosure are administered at 0.4 mg/kg. In some embodiments. ActRII polypeptides of the disclosure are administered at 0.5 mg/kg. In some embodiments, ActRII polypeptides of the disclosure are administered at 0.6 mg/kg. In some embodiments, ActRII polypeptides of the disclosure are administered at 0.7 mg/kg. In some embodiments, ActRII polypeptides of the disclosure are administered at 0.8 mg/kg. In some embodiments, ActRII polypeptides of the disclosure are administered at 0.9 mg/kg. In some embodiments, ActRII polypeptides of the disclosure are administered at 1.0 mg/kg. In some embodiments, ActRII polypeptides of the disclosure are administered at 1.1 mg/kg. In some embodiments. ActRII polypeptides of the disclosure are administered at 1.2 mg/kg. In some embodiments, ActRII polypeptides of the disclosure are administered at 1.25 mg/kg. In some embodiments, ActRII polypeptides of the disclosure are administered at 1.3 mg/kg. In some embodiments, ActRII polypeptides of the disclosure are administered at 1.33 mg/kg. In some embodiments, ActRII polypeptides of the disclosure are administered at 1.4 mg/kg. In some embodiments, ActRII polypeptides of the disclosure are administered at 1.5 mg/kg. In some embodiments, ActRII polypeptides of the disclosure are administered at 1.6 mg/kg. In some embodiments. ActRII polypeptides of the disclosure are administered at 1.7 mg/kg. In some embodiments. ActRII polypeptides of the disclosure are administered at 1.75 mg/kg. In some embodiments, ActRII polypeptides of the disclosure are administered at 1.8 mg/kg. In some embodiments, ActRII polypeptides of the disclosure are administered at 1.9 mg/kg. In some embodiments, ActRII polypeptides of the disclosure are administered at 2.0 mg/kg.

The disclosure also provides formulations that may be varied to include acids and bases to adjust the pH; and buffering agents to keep the pH within a narrow range.

4. Kits

The present disclosure provides a kit comprising a lyophilized polypeptide and an injection device. In certain embodiments, the lyophilized polypeptide comprises an ActRII polypeptide (e.g., a polypeptide that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 2 or SEQ ID NO: 3), or fragments, functional variants, or modified forms thereof. In certain embodiments, the polypeptide binds to one or more ligands selected from the group consisting of activin A, activin B, and GDF11. In certain such embodiments, the polypeptide further binds to one or more ligands selected from the group consisting of BMP10, GDF8, and BMP6. In certain embodiments, the polypeptide binds to activin and/or GDF11.

In some embodiments, the lyophilized polypeptide comprises a polypeptide that comprises, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 2 or SEQ ID NO: 3. In certain such embodiments, the polypeptide comprises an amino acid sequence that is least 90%, 95%, or 99% identical to SEQ ID NO: 2 or SEQ ID NO: 3, wherein the polypeptide binds to activin and/or GDF11. In certain embodiments, the polypeptide comprises the amino acid sequence of SEQ ID NO: 2 or SEQ ID NO: 3. In other embodiments, the polypeptide consists of the amino acid sequence of SEQ ID NO: 2 or SEQ ID NO: 3.

In some embodiments, the lyophilized polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 3. In certain embodiments, the polypeptide consists essentially of the amino acid sequence of SEQ ID NO: 3. In other embodiments, the polypeptide consists of the amino acid sequence of SEQ ID NO: 3.

In certain embodiments of the foregoing, the lyophilized polypeptide comprises a fusion protein further comprising an Fc domain of an immunoglobulin. In certain such embodiments, the Fc domain of the immunoglobulin is an Fc domain of an IgG1 immunoglobulin. In other embodiments, the fusion protein further comprises a linker domain positioned between the polypeptide domain and the Fc domain of the immunoglobulin. In certain embodiments, the linker domain is a polyglycine linker.

In certain embodiments, the lyophilized polypeptide is part of a homodimer protein complex.

In certain embodiments, the polypeptide is glycosylated.

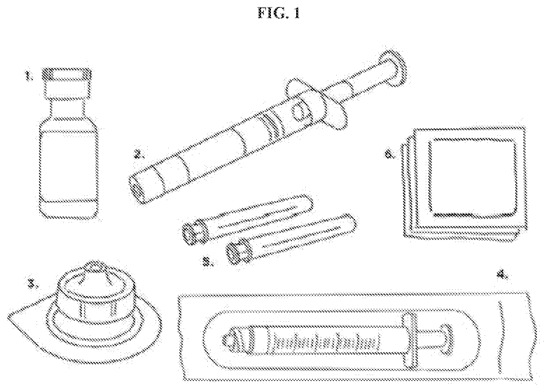

The present disclosure provides a kit comprising a sterile pow-der comprising a lyophilized polypeptide as disclosed herein and an injection device. In some embodiments of the kits disclosed herein, the sterile powder comprising a lyophilized polypeptide is pre-filled in one or more containers, such as one or more vials FIG. 1.

In certain embodiments, the pH range for the sterile powder comprising a lyophilized polypeptide is from 6 to 8. In some embodiments, the sterile powder comprising a lyophilized polypeptide further comprises a buffering agent. In some embodiments, the buffering agent may be added in an amount of at least 10 mM. In some embodiments, the buffering agent may be added in an amount in the range of between about 10 mM to about 200 mM. In some embodiments, the buffering agent comprises citric acid monohydrate and/or tri-sodium citrate dehydrate.

In some embodiments, the sterile powder comprising a lyophilized polypeptide further comprises a surfactant. In some embodiments, the surfactant comprises a polysorbate. In some embodiments, the surfactant comprises polysorbate 80.

In some embodiments, the sterile powder comprising a lyophilized polypeptide further comprises a lyoprotectant. In some embodiments, the lyoprotectant comprises a sugar, such as disaccharides (e.g., sucrose). In some embodiments, the lyoprotectant comprises sucrose, trehalose, mannitol, polyvinylpyrrolidone (PVP), dextrose, and/or glycine. In some embodiments, the lyoprotectant comprises sucrose. In some embodiments, the sterile powder comprises the lyoprotectant and lyophilized polypeptide in a weight ratio of at least 1:1 lyophilized polypeptide to lyoprotectant. In some embodiments, the sterile powder comprises the lyoprotectant and lyophilized polypeptide in a weight ratio of from 1:1 to 1:10 lyophilized polypeptide to lyoprotectant. In some embodiments, the sterile powder comprises the lyoprotectant and lyophilized polypeptide in a weight ratio of 1:1, 1:2, 1:3, 1:4, 1:5, 1:6, 1:7, 1:8, 1:9, or 1:10 lyophilized polypeptide to lyoprotectant. In some embodiments, the sterile powder comprises the lyoprotectant and lyophilized polypeptide in a weight ratio of 1:6 lyophilized polypeptide to lyoprotectant. In certain embodiments of the foregoing, the sterile powder comprises lyoprotectant in an amount sufficient to stabilize the lyophilized polypeptide.

In certain embodiments of the kits disclosed herein, the injection device comprises a syringe (FIG. 1). In certain such embodiments, the syringe is pre-filled with a reconstitution solution. In some embodiments, the reconstitution solution comprises a pharmaceutically acceptable carrier and/or excipient. In some embodiments, the pharmaceutically acceptable carrier comprises aqueous solutions such as water, physiologically buffered saline, or other solvents or vehicles such as glycols, glycerol, oils or injectable organic esters. In some embodiments, the pharmaceutically acceptable excipient comprises a pharmaceutically acceptable excipient selected from calcium phosphates, calcium carbonates, calcium sulfates, halites, metallic oxides, sugars, sugar alcohols, starch, glycols, povidones, mineral hydrocarbons, acrylic polymers, fatty alcohols, mineral stearates, glycerin, and/or lipids. In certain embodiments, the reconstitution solution comprises pharmaceutically acceptable sterile isotonic aqueous or nonaqueous solutions, dispersions, suspensions or emulsions. In certain such embodiments, the reconstitution solution comprises antioxidants, buffers, bacteriostats, and/or solutes which render the formulation isotonic with the blood of the intended recipient. In other embodiments, the reconstitution solution comprises suspending or thickening agents.

In certain embodiments of the kits disclosed herein, the kit further comprises a vial adapter (FIG. 1). In some embodiments, the vial pre-filled with sterile powder comprising a lyophilized polypeptide attaches to one end of the vial adapter. In some embodiments, the syringe pre-filled with a reconstitution solution as disclosed herein attaches to an end of the vial adapter. In some embodiments, the syringe pre-filled with a reconstitution solution as disclosed herein and the vial pre-filled with sterile powder comprising a lyophilized polypeptide are attached to opposite ends of the vial adapter. In some embodiments, the reconstitution solution is transferred from the pre-filled syringe to the vial. In some embodiments, transfer of the reconstitution solution to the vial pre-filled with sterile powder comprising a lyophilized polypeptide reconstitutes the lyophilized polypeptide into a sterile injectable solution. In some embodiments, the lyophilized polypeptide is reconstituted into a sterile injectable solution. In some embodiments, the lyophilized polypeptide is reconstituted into a sterile injectable solution prior to use.

In other embodiments of the kits disclosed herein, the kit further comprises a pump apparatus. In certain embodiments, the pump apparatus comprises an electromechanical pumping assembly. In certain embodiments, the pump apparatus comprises a reservoir for holding a sterile injectable solution. In certain embodiments, the reservoir holds 1 mL of sterile injectable solution. In certain embodiments, the pump apparatus comprises one or more vials or cartridges comprising a sterile injectable solution. In certain embodiments, the vials or cartridges are prefilled with sterile injectable solution. In certain embodiments, the vials or cartridges comprise sterile injectable solution reconstituted from a lyophilized polypeptide. In certain embodiments, the reservoir is coupled to the vial or cartridge. In certain embodiments, the vial or cartridge holds 1-20 mL of sterile injectable solution. In certain embodiments, the electromechanical pumping assembly comprises a pump chamber. In certain embodiments, the electromechanical pumping assembly is coupled to the reservoir. In certain embodiments, the sterile injectable solution is received from the reservoir into the pump chamber. In some embodiments, the electromechanical pumping assembly comprises a plunger that is disposed such that sterile injectable solution in the pump chamber is in direct contact with the plunger. In certain embodiments, a sterile injectable solution is received from the reservoir into the pump chamber during a first pumping phase, and is delivered from the pump chamber to a subject during a second pumping phase. In certain embodiments, the electromechanical pumping assembly comprises control circuitry. In certain embodiments, control circuitry drives the plunger to (a) draw the sterile injectable solution into the pump chamber during the first pumping phase and (b) deliver the sterile injectable solution from the pump chamber in a plurality of discrete motions of the plunger during the second pumping phase, thereby delivering the therapeutic substance to the subject in a plurality of controlled and discrete dosages throughout the second pumping phase. In certain embodiments, a cycle of alternating the first and second pumping phases may be repeated until a desired dose is administered. In certain embodiments, the pump apparatus is coupled to a wearable patch. In certain embodiments, the pump apparatus is a wearable pump apparatus.

The present disclosure provides a kit used for reconstituting a lyophilized polypeptide into a sterile injectable solution. In certain embodiments, the resulting sterile injectable solution is useful in the methods disclosed herein.

In certain embodiments of the kits disclosed herein, the kit further comprises an injectable device for use in administering the sterile injectable solution parenterally FIG. 1. In some embodiments, the sterile injectable solution is administered via subcutaneous injection. In some embodiments, the sterile injectable solution is administered via intradermal injection. In some embodiments, the sterile injectable solution is administered via intramuscular injection. In some embodiments, the sterile injectable solution is administered via intravenous injection. In some embodiments, the sterile injectable solution is self-administered. In some embodiments, the sterile injectable solution comprises a therapeutically effective dose. In some embodiments, the therapeutically effective dose comprises a weight based dose.

5. ActRII Polypeptides

In certain aspects, the disclosure relates to the dosing regimens, dosage forms, and formulations comprising ActRIIB Ligand Trap polypeptides, e.g., soluble variant ActRIIB polypeptides, including, for example, fragments, functional variants, and modified forms of ActRIIB polypeptides. In certain embodiments, the ActRIIB Ligand Trap polypeptides have at least one similar or same biological activity as a corresponding wild-type ActRIIB polypeptide. For example, a ActRIIB Ligand Trap polypeptide of the disclosure may bind to and inhibit the function of an ActRIIB ligand (e.g., activin A, activin AB, activin B. Nodal, GDF8, GDF11 or BMP7). Examples of ActRIIB Ligand Trap polypeptides include human ActRIIB precursor polypeptides (SEQ ID NO: 1) having one or more sequence variations, and soluble human ActRIIB polypeptides (e.g., SEQ ID NOs: 2) having one or more sequence variations. A ActRIIB Ligand Trap refers to an ActRIIB polypeptide having a decreased affinity for activin relative to other ActRIIB ligands, including for example GDF11 and/or myostatin.

As used herein, the term “ActRIIB” refers to a family of activin receptor type 11B (ActRIIB) proteins from any species and variants derived from such ActRIIB proteins by mutagenesis or other modification. Reference to ActRIIB herein is understood to be a reference to any one of the currently identified forms. Members of the ActRIIB family are generally transmembrane proteins, composed of a ligand-binding extracellular domain with a cysteine-rich region, a transmembrane domain, and a cytoplasmic domain with predicted serine/threonine kinase activity.

The term “ActRIIB polypeptide” includes polypeptides comprising any naturally occurring polypeptide of an ActRIIB family member as well as any variants thereof (including mutants, fragments, fusions, and peptidomimetic forms) that retain a useful activity. See, for example, WO 2006/012627. For example, ActRIIB polypeptides include polypeptides derived from the sequence of any known ActRIIB having a sequence at least about 80% identical to the sequence of an ActRIIB polypeptide, and optionally at least 85%, 90%, 95%, 97%, 99% or greater identity. For example, an ActRIIB polypeptide may bind to and inhibit the function of an ActRIIB protein and/or activin. An ActRIIB polypeptide which is a ActRIIB Ligand Trap may be selected for activity in promoting red blood cell formation in vivo. Examples of ActRIIB polypeptides include human ActRIIB precursor polypeptide (SEQ ID NO: 1) and soluble human ActRIIB polypeptides (e.g., SEQ ID NO: 3). Numbering of amino acids for all ActRIIB-related polypeptides described herein is based on the numbering for SEQ ID NO: 1, unless specifically designated otherwise.

The human ActRIIB precursor protein sequence is as follows:

(SEQ ID NO: 1)

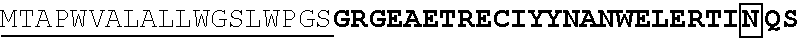

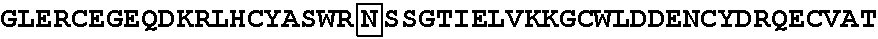

EENPQVYFCCCEGNFCNERFTHLPEAGGPEVTYEPPPTAPTLLTVLAYS
LLPIGGLSLIVLLAFWMYRHRKPPYGHVDIHEDPGPPPPSPLVGLKPLQ
LLEIKARGREGCVWKAQLMNDFVAVKIFPLQDKQSWQSEREIFSTPGMK
HENLLQFIAAEKRGSNLEVELWLITAFHDKGSLTDYLKGNIITWNELCH
VAETMSRGLSYLHEDVPWCRGEGHKPSIAHRDFKSKNVILKSDLTAVLA
DEGLAVRFEPGKPPGDTHGQVGTRRYMAPEVLEGAINFORDAFLRIDMY
AMGLVLWELVSRCKAADGPVDEYMLPFEEEIGQHPSLEELQEVVVHKKM
RPTIKDHWLKHPGLAQLCVTIEECWDHDAEARLSAGCVEERVSLIRRSV
NGTTSDCLVSLVTSVTNVDLPPKESSI

The signal peptide is single underlined; the extracellular domain is in bold and the potential N-linked glycosylation sites are in boxes.

The human ActRIIB soluble (extracellular), processed polypeptide sequence is as follows:

(SEQ ID NO: 2)
GRGEAETRECIYYNANWELERTNQSGLERCEGEQDKRLHCYASW
RNSSGTIELVKKGCWLDDENCYDRQECVATEENPQVYFCCCEGNFCNER
FTHLPEAGGPEVTYEPPPTAPT

In a specific embodiment, the disclosure relates to ActRIIB Ligand Trap polypeptides which are variant forms of soluble ActRIIB polypeptides. As described herein, the term “soluble ActRIIB polypeptide” generally refers to polypeptides comprising an extracellular domain of an ActRIIB protein. The term “soluble ActRIIB polypeptide.” as used herein, includes any naturally occurring extracellular domain of an ActRIIB protein as well as any variants thereof (including mutants, fragments and peptidomimetic forms) that retain a useful activity. For example, the extracellular domain of an ActRIIB protein binds to a ligand and is generally soluble. Examples of soluble ActRIIB polypeptides include ActRIIB soluble polypeptides (e.g., SEQ ID NOs: 3). Other examples of soluble ActRIIB polypeptides comprise a signal sequence in addition to the extracellular domain of an ActRIIB protein. The signal sequence can be a native signal sequence of an ActRIIB, or a signal sequence from another protein, such as a tissue plasminogen activator (TPA) signal sequence or a honey bee melittin (HBM) signal sequence.

The disclosure identifies functionally active portions and variants of ActRIIB. Applicants have ascertained that an Fc fusion protein having the sequence disclosed by Hilden et al. (Blood. 1994 Apr. 15; 83(8):2163-70), which has an Alanine at the position corresponding to amino acid 64 of SEQ ID NO: 1 (A64), has a relatively low affinity for activin and GDF-11. By contrast, the same Fc fusion protein with an Arginine at position 64 (R64) has an affinity for activin and GDF-11 in the low nanomolar to high picomolar range. Attisano et al. (Cell. 1992 Jan. 10; 68(1):97-108) showed that a deletion of the proline knot at the C-terminus of the extracellular domain of ActRIIB reduced the affinity of the receptor for activin. An ActRIIB-Fc fusion protein containing amino acids 20-119 of SEQ ID NO: 1, “ActRIIB(20-119)-Fc”, has reduced binding to GDF-11 and activin relative to an ActRIIB(20-134)-Fc, which includes the proline knot region and the complete juxtamembrane domain. However, an ActRIIB(20-129)-Fc protein retains similar but somewhat reduced activity relative to the wild type, even though the proline knot region is disrupted. Thus, ActRIIB extracellular domains that stop at least at at amino acid 134, 133, 132, 131, 130 and 129 are all expected to be active. Therefore, a ActRIIB Ligand Trap polypeptide which is an ActRIIB-Fc fusion protein may end as early as amino acid 109 (the final cysteine), however, forms ending at or between 109 and 119 are expected to have reduced ligand binding. Amino acid 119 is poorly conserved and so is readily altered or truncated. Forms ending at 128 or later retain ligand binding activity. Forms ending at or between 119 and 127 will have an intermediate binding ability. Any of these forms may be desirable to use, depending on the clinical or experimental setting.

At the N-terminus of ActRIIB, it is expected that a protein beginning at amino acid 29 or before will retain ligand binding activity. Amino acid 29 represents the initial cysteine. An alanine to asparagine mutation at position 24 introduces an N-linked glycosylation sequence without substantially affecting ligand binding. This confirms that mutations in the region between the signal cleavage peptide and the cysteine cross-linked region, corresponding to amino acids 20-29 are well tolerated. In particular, constructs beginning at position 20, 21, 22, 23 and 24 will retain activity, and constructs beginning at positions 25, 26, 27, 28 and 29 are also expected to retain activity.

Taken together, an active portion of ActRIIB comprises amino acids 29-109 of SEQ ID NO: 1, and ActRIIB Ligand Trap constructs may, for example, comprise a portion of ActRIIB beginning at a residue corresponding to amino acids 20-29 of SEQ ID NO: 1 and ending at a position corresponding to amino acids 109-134 of SEQ ID NO: 1. Other examples include constructs that begin at a position from 20-29 or 21-29 and end at a position from 119-134, 119-133, 129-134, or 129-133 of SEQ ID NO: 1. Other examples include constructs that begin at a position from 20-24 (or 21-24, or 22-25) and end at a position from 109-134 (or 109-133), 119-134 (or 119-133) or 129-134 (or 129-133) of SEQ ID NO: 1. Variants within these ranges are also contemplated, particularly those having at least 80%, 85%, 90%, 95% or 99% identity to the corresponding portion of SEQ ID NO: 1. In certain embodiments, the ActRIIB Ligand Trap polypeptide comprises, consists essentially of, or consists of, a polypeptide having an amino acid sequence that is at least 80%, 85%, 90%, 95%, 96%, 97%, 98%, 99% or 100% identical to amino acid residues 25-131 of SEQ ID NO: 1. In certain embodiments, the ActRIIB Ligand Trap polypeptide comprises, consists essentially of, or consists of, a polypeptide having an amino acid sequence that is at least 80%, 85%, 90%, 95%, 96%, 97%, 98%, 99% or 100% identical to SEQ ID NOs: 3. In preferred embodiments, the ActRIIB Ligand Trap polypeptide consists of, or consists essentially of, the amino acid sequence of SEQ ID NO: 3.

Position L79 of ActRIIB may be altered to confer altered activin-myostatin (GDF-11) binding properties. L79A or L79P reduces GDF-11 binding to a greater extent than activin binding. L79E or L79D retains GDF-11 binding. Remarkably, the L79E and L79D variants have greatly reduced activin binding. In vivo experiments indicate that these non-activin receptors retain significant ability to increase red blood cells but show decreased effects on other tissues. These data demonstrate the desirability and feasibility for obtaining polypeptides with reduced effects on activin. In exemplary embodiments, the methods described herein utilize a ActRIIB Ligand Trap polypeptide which is a variant ActRIIB polypeptide comprising an acidic amino acid (e.g., D or E) at the position corresponding to position 79 of SEQ ID NO: 1, optionally in combination with one or more additional amino acid substitutions, additions, or deletions.

In certain embodiments, the ActRIIB Ligand Trap polypeptides of the disclosure may further comprise post-translational modifications in addition to any that are naturally present in the ActRIIB polypeptides. Such modifications include, but are not limited to, acetylation, carboxylation, glycosylation, phosphorylation, lipidation, and acylation. As a result, ActRIIB Ligand Trap polypeptides may contain non-amino acid elements, such as polyethylene glycols, lipids, poly- or mono-saccharide, and phosphates. Effects of such non-amino acid elements on the functionality of a ActRIIB Ligand Trap polypeptide may be tested as described herein for other ActRIIB Ligand Trap polypeptide variants. When a ActRIIB Ligand Trap polypeptide is produced in cells by cleaving a nascent form of the ActRIIB Ligand Trap polypeptide, post-translational processing may also be important for correct folding and/or function of the protein. Different cells (such as CHO, HeLa, MDCK, 293, WI38, NIH-3T3 or HEK293) have specific cellular machinery and characteristic mechanisms for such post-translational activities and may be chosen to ensure the correct modification and processing of the ActRIIB Ligand Trap polypeptides.

In certain embodiments, the ActRIIB Ligand Trap polypeptides of the present disclosure contain one or more modifications that are capable of stabilizing the ActRIIB Ligand Trap polypeptides. For example, such modifications enhance the in vitro half life of the ActRIIB Ligand Trap polypeptides, enhance circulatory half life of the ActRIIB Ligand Trap polypeptides or reducing proteolytic degradation of the ActRIIB Ligand Trap polypeptides. Such stabilizing modifications include, but are not limited to, fusion proteins (including, for example, fusion proteins comprising an ActRIIB Ligand Trap polypeptide and a stabilizer domain), modifications of a glycosylation site (including, for example, addition of a glycosylation site to a ActRIIB Ligand Trap polypeptide), and modifications of carbohydrate moiety (including, for example, removal of carbohydrate moieties from a ActRIIB Ligand Trap polypeptide). In the case of fusion proteins, a ActRIIB Ligand Trap polypeptide is fused to a stabilizer domain such as an IgG molecule (e.g., an Fc domain). As used herein, the term “stabilizer domain” not only refers to a fusion domain (e.g., Fc) as in the case of fusion proteins, but also includes nonproteinaceous modifications such as a carbohydrate moiety, or nonproteinaceous polymer, such as polyethylene glycol.

In certain embodiments, the present disclosure makes available isolated and/or purified forms of the ActRIIB Ligand Trap polypeptides, which are isolated from, or otherwise substantially free of, other proteins.

In certain embodiments, ActRIIB Ligand Trap polypeptides (unmodified or modified) of the disclosure can be produced by a variety of art-known techniques. For example, such ActRIIB Ligand Trap polypeptides can be synthesized using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company. New York (1992). In addition, automated peptide synthesizers are commercially available (e.g., Advanced ChemTech Model 396; Milligen/Biosearch 9600). Alternatively, the ActRIIB Ligand Trap polypeptides, fragments or variants thereof may be recombinantly produced using various expression systems (e.g., E. coli, Chinese Hamster Ovary (CHO) cells, COS cells, baculovirus) as is well known in the art. In a further embodiment, the modified or unmodified ActRIIB Ligand Trap polypeptides may be produced by digestion of recombinantly produced full-length ActRIIB Ligand Trap polypeptides by using, for example, a protease, e.g., trypsin, thermolysin, chymotrypsin, pepsin, or paired basic amino acid converting enzyme (PACE). Computer analysis (using a commercially available software, e.g., MacVector, Omega, PCGene, Molecular Simulation, Inc.) can be used to identify proteolytic cleavage sites. Alternatively, such ActRIIB Ligand Trap polypeptides may be produced from recombinantly produced full-length ActRIIB Ligand Trap polypeptides such as standard techniques known in the art, such as by chemical cleavage (e.g., cyanogen bromide, hydroxylamine).

In certain embodiments of the dosing regimens, dosage forms, and formulations provided herein, a soluble ActRII polypeptide is formulated as a lyophilized polypeptide formulation comprising a therapeutic amount of a soluble ActRII polypeptide disclosed herein, whereby the lyophilized polypeptide formulation is reconstitutable to a solution in liquid form. In some embodiments, the lyophilized polypeptide formulation is reconstituted in a sterile injectable solution or a reconstitution solution. In some embodiments, the sterile injectable solution or reconstitution solution comprises a pharmaceutically acceptable carrier, excipient, and/or additive. In some embodiments, the sterile injectable solution or reconstitution solution comprises saline solution, purified water, or sterile water for injection.

In certain embodiments, the biological activity of the ActRII polypeptide in the dosing regimens, dosage forms, and formulations provided herein can be characterized by bioassays such as binding to TGFβ ligand family members, effector function potential, and binding to neonatal receptor (FcRn).

In certain embodiments, the ActRII polypeptide in the dosing regimens, dosage forms, and formulations provided herein can be a potent inhibitor of GDF-11 dependent activation of ActRIIB receptor SMAD2/3 signaling and can act as an erythroid maturation agent.

In certain embodiments, the ActRII polypeptide in the dosing regimens, dosage forms, and formulations provided herein can selectively bind to GDF-11 and GDF-8, but can have reduced affinity to other TGFβ ligand family members that bind to ACTIIB receptor.

In certain embodiments, the ActRII polypeptide in the dosing regimens, dosage forms, and formulations provided herein can act as a soluble ligand trap and does not induce effector function.

In certain embodiments, the ActRII polypeptide in the dosing regimens, dosage forms, and formulations provided herein can be associated with FcRn such that the association is consistent with that of an Fe containing protein and competed for binding to a wild-type human IgG1 antibody. Any suitable assay known to the skilled artisan can be used to demonstrate these activities.

6. Polynucleotides

In certain aspects, the disclosure provides isolated and/or recombinant nucleic acids encoding any of the ActRIIB Ligand Trap polypeptides disclosed herein. SEQ ID NOs: 4 encodes a soluble ActRIIB Ligand Trap. The subject nucleic acids may be single-stranded or double stranded. Such nucleic acids may be DNA or RNA molecules. These nucleic acids may be used, for example, in methods for making ActRIIB Ligand Trap polypeptides.

In certain embodiments, the disclosure provides isolated or recombinant nucleic acid sequences that are at least 80%, 85%, 90%, 95%, 97%, 98%, 99% or 100% identical to SEQ ID NO: 4. One of ordinary skill in the art will appreciate that nucleic acid sequences complementary to SEQ ID NO: 4, are also within the scope of this disclosure. In further embodiments, the nucleic acid sequences of the disclosure can be isolated, recombinant, and/or fused with a heterologous nucleotide sequence, or in a DNA library.

In other embodiments, nucleic acids of the disclosure also include nucleotide sequences that hybridize under highly stringent conditions to the nucleotide sequence designated in SEQ ID NO: 4, complement sequence of SEQ ID NO: 4, or fragments thereof. As discussed above, one of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ from the nucleic acids as set forth in SEQ ID NO: 4 due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. In certain embodiments, the ActRIIB Ligand Trap polypeptide will be encoded by an alternative nucleotide sequence. Alternative nucleotide sequences are degenerate with respect to the native ActRIIB Ligand Trap nucleic acid sequence but still encode for the same fusion protein. In certain embodiments, the ActRIIB Ligand Trap having SEQ ID NO: 3 is encoded by an alternative nucleic acid sequence. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

In certain embodiments, the recombinant nucleic acids of the disclosure may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In a preferred embodiment, the expression vector contains a selectable marker gene to allow the selection of transformed host cells. Selectable marker genes are well known in the art and will vary with the host cell used.

In certain aspects of the disclosure, the subject nucleic acid is provided in an expression vector comprising a nucleotide sequence encoding a ActRIIB Ligand Trap polypeptide and operably linked to at least one regulatory sequence. Regulatory sequences are art-recognized and are selected to direct expression of the ActRIIB Ligand Trap polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding a ActRIIB Ligand Trap polypeptide. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid of the disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant ActRIIB Ligand Trap polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the ß-gal containing pBlueBac III).

In a preferred embodiment, a vector will be designed for production of the subject ActRIIB Ligand Trap polypeptides in CHO cells, such as a Pcmv-Script vector (Stratagene, La Jolla, Calif.), pcDNA4 vectors (Invitrogen, Carlsbad, Calif.) and pCI-neo vectors (Promega, Madison. Wis.). As will be apparent, the subject gene constructs can be used to cause expression of the subject ActRIIB Ligand Trap polypeptides in cells propagated in culture, e.g., to produce proteins, including fusion proteins or variant proteins, for purification.

This disclosure also pertains to a host cell transfected with a recombinant gene including a coding sequence (e.g., SEQ ID NO: 4) for one or more of the subject ActRIIB Ligand Trap polypeptides. The host cell may be any prokaryotic or eukaryotic cell. For example, a ActRIIB Ligand Trap polypeptide of the disclosure may be expressed in bacterial cells such as E. coli, insect cells (e.g., using a baculovirus expression system), yeast, or mammalian cells. Other suitable host cells are known to those skilled in the art.

Accordingly, the present disclosure further pertains to methods of producing the subject ActRIIB Ligand Trap polypeptides. For example, a host cell transfected with an expression vector encoding a ActRIIB Ligand Trap polypeptide can be cultured under appropriate conditions to allow expression of the ActRIIB Ligand Trap polypeptide to occur. The ActRIIB Ligand Trap polypeptide may be secreted and isolated from a mixture of cells and medium containing the ActRIIB Ligand Trap polypeptide. Alternatively, the ActRIIB Ligand Trap polypeptide may be retained cytoplasmically or in a membrane fraction and the cells harvested, lysed and the protein isolated. A cell culture includes host cells, media and other byproducts. Suitable media for cell culture are well known in the art. The subject ActRIIB Ligand Trap polypeptides can be isolated from cell culture medium, host cells, or both, using techniques known in the art for purifying proteins, including ion-exchange chromatography, gel filtration chromatography, ultrafiltration, electrophoresis, and immunoaffinity purification with antibodies specific for particular epitopes of the ActRIIB Ligand Trap polypeptides. In a preferred embodiment, the ActRIIB Ligand Trap polypeptide is a fusion protein containing a domain which facilitates its purification.

In another embodiment, a fusion gene coding for a purification leader sequence, such as a poly-(His)/enterokinase cleavage site sequence at the N-terminus of the desired portion of the recombinant ActRIIB Ligand Trap polypeptide, can allow purification of the expressed fusion protein by affinity chromatography using a Ni²⁺ metal resin. The purification leader sequence can then be subsequently removed by treatment with enterokinase to provide the purified ActRIIB Ligand Trap polypeptide (e.g., see Hochuli et al., (1987) J. Chromatography 411:177; and Janknecht et al., PNAS USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

### 7. Exemplary Uses

In certain embodiments, the dosage forms comprising the ActRIIB Ligand Trap described herein can be used for treating a human subject with anemia associated with a thalassemia. Thalassemia syndromes are hereditary hemoglobinopathies in which imbalances in the production of intact alpha- and beta-hemoglobin chains lead to increased apoptosis during erythroblast maturation [see, e.g., Schrier (2002) Curr Opin Hematol 9:123-126]. Thalassemias are collectively among the most frequent genetic disorders worldwide, with changing epidemiologic patterns predicted to contribute to a growing public health problem in both the U.S. and globally [Vichinsky (2005) Ann NY Acad Sci 1054:18-24]. Thalassemia syndromes are named according to their severity. Thus, α-thalassemias include α-thalassemia minima. α-thalassemia minor (also known as α-thalassemia trait: two affected α-globin genes), hemoglobin H disease (three affected α-globin genes), and α-thalassemia major (also known as hydrops fetalis: four affected α-globin genes). β-Thalassemias include β-thalassemia minor (also known as β-thalassemia trait: one affected β-globin gene), β-thalassemia intermedia (two affected β-globin genes), hemoglobin E thalassemia (two affected β-globin genes), and β-thalassemia major (also known as Cooley's anemia; two affected β-globin genes resulting in a complete absence of β-globin protein). β-Thalassemia impacts multiple organs, is associated with considerable morbidity and mortality, and currently requires life-long care. Although life expectancy in patients with β-thalassemia has increased in recent years due to use of regular blood transfusions in combination with iron chelation, iron overload resulting both from transfusions and from excessive gastrointestinal absorption of iron can cause serious complications such as heart disease, thrombosis, hypogonadism, hypothyroidism, diabetes, osteoporosis, and osteopenia Isee, e.g., Rund et al. (2005) N Engl J Med 353:1135-11461. δ-β Thalassemia is a form of β-thalassemia characterized by decreased or absent synthesis of the delta- and beta-globin chains with a compensatory increase in expression of fetal gamma-chain synthesis. Subjects heterozygous for δ-β thalassemia are clinically asymptomatic, but subjects homozygous for δ-β thalassemia have mild clinical presentation.

In some embodiments, the dosage forms of the disclosure can be used for treating anemia associated with thalassemia. In some embodiments, the thalassemia is alpha-thalassemia. In some embodiments, the alpha-thalassemia is alpha-thalassemia minima. In some embodiments, the alpha-thalassemia is alpha-thalassemia-minor. In some embodiments, the alpha-thalassemia is Hemoglobin H disease. In some embodiments, the alpha-thalassemia is alpha-thalassemia-major. In some embodiments, the thalassemia is beta-thalassemia. In some embodiments, the beta-thalassemia is beta-thalassemia minor. In some embodiments, the beta-thalassemia is beta-thalassemia intermedia. In some embodiments, the beta-thalassemia is beta-thalassemia major. In some embodiments, the beta-thalassemia is Hemoglobin E disease. In some embodiments, the thalassemia is delta-beta-thalassemia.

In certain embodiments, the formulations comprising the ActRIIB Ligand Trap described herein can be used for treating a human subject diagnosed with anemia due to very low, low, or intermediate risk myelodysplastic syndromes (MDS).

The subjects treated in accordance with the methods described herein can be any mammals such as rodents and primates, and in a preferred embodiment, humans. In certain embodiments, the methods described herein can be used to treat anemia due to very low, low, or intermediate risk Myelodysplastic syndromes (MDS) in a subject, to reduce transfusion burden in a subject with anemia, or to monitor said treatment, and/or to select subjects to be treated in accordance with the dosage forms and methods provided herein, in any mammal such as a rodent or primate, and in a preferred embodiment, in a human subject.

In certain embodiments, the subject treated in accordance with the methods described herein is female. In certain embodiments, the subject treated in accordance with the methods described herein is male. In certain embodiments, the subject treated in accordance with the methods described herein can be of any age. In certain embodiments, the subject treated in accordance with the methods described herein is less than 18 years old. In a specific embodiment, the subject treated in accordance with the methods described herein is less than 13 years old. In another specific embodiment, the subject treated in accordance with the methods described herein is less than 12, less than 11, less than 10, less than 9, less than 8, less than 7, less than 6, or less than 5 years old. In another specific embodiment, the subject treated in accordance with the methods described herein is 1-3 years old, 3-5 years old, 5-7 years old, 7-9 years old, 9-11 years old, 11-13 years old, 13-15 years old, 15-20 years old, 20-years old, 25-30 years old, or greater than 30 years old. In another specific embodiment, the subject treated in accordance with the methods described herein is 30-35 years old, 35-40 years old, 40-45 years old, 45-50 years old, 50-55 years old, 55-60 years old, or greater than 60 years old. In another specific embodiment, the subject treated in accordance with the methods described herein is 18-64 years old, 65-74 years old, or greater than 75 years old.

In certain embodiments, a subject treated in accordance with the dosage forms and methods provided herein has been diagnosed with IPSS-R defined MDS. IPSS-R refers to the International Prognostic Scoring System-Revised, which is utilized in the evaluation of prognosis in myelodysplastic syndromes. See, e.g., Greenberg et al., Blood, 120(12):2454-2465 (2012). The IPSS-R utilizes a criteria point system to characterize myelodysplastic syndrome patient outcomes as very low risk (0-1.5 risk score, median survival 8.8 years), low risk (1.5-3.0 risk score; median survival of 5.3 years), intermediate (3.0-4.5 point; median survival of 3.0 years); high risk (4.5-6.0 points; median survival of 1.6 years); or very high risk (risk score higher than 6; median survival of 0.8 years). The point system evaluates (i) the percentage of bone marrow blasts in the subject; and (ii) cytogenetics in the subject which defined as hemoglobin concentration (g/dL), absolute neutrophil count (×10⁹/L), and platelet count (×10⁹/L).

In certain embodiments, a subject treated in accordance with the dosage forms and methods provided herein has MDS. In certain embodiments, the MDS is IPSS-defined very low risk MDS. In certain embodiments, the MDS is IPSS-R defined low risk MDS. In certain embodiments, the MDS is IPSS-R defined intermediate risk MDS. In certain embodiments, a subject treated in accordance with the dosage forms and methods provided herein has MDS-refractory cytopenia with multilineage dysplasia (MDS-RCMD).

In certain embodiments, the subject treated in accordance with the methods described herein has an Eastern Cooperative Oncology Group (ECOG) score of 0. In certain embodiments, the subject treated in accordance with the methods described herein has an ECOG score of 1. In certain embodiments, the subject treated in accordance with the methods described herein has an ECOG score of 2.

In certain embodiments, the percentage of erythroblasts in a subject treated in accordance with the dosage forms and methods provided herein that are ring sideroblasts is at least 5%, 6%, 7%, 8%, 9%, 10%, 11%, 12%, 13%, 14%, 15%, 16%, 17%, 18%, 19%, or at least 20%. In certain embodiments, the percentage of erythroblasts in a subject treated in accordance with the dosage forms and methods provided herein that are ring sideroblasts is at least 15%. In certain embodiments, the percentage of erythroblasts in a subject treated in accordance with the dosage forms and methods provided herein that are ring sideroblasts is about 15%. In certain embodiments, the percentage of erythroblasts in a subject treated in accordance with the dosage forms and methods provided herein that are ring sideroblasts is between about 15% and about 20%. In certain embodiments, the percentage of erythroblasts in a subject treated in accordance with the dosage forms and methods provided herein that are ring sideroblasts is between about 5% and 20%. In certain embodiments, a subject treated in accordance with the dosage forms and methods provided herein has a ringed sideroblast to normal erythroblast ratio of at least 1:20, at least 1:7, or at least 1:5.

In certain embodiments, a subject having anemia due to very low, low, or intermediate risk MDS treated requires regular, lifelong red blood cell transfusions. In certain embodiments, a subject having anemia due to very low, low, or intermediate risk MDS requires transfusion of 0 to 4 red blood cell units over a 8-weeks period. In certain embodiments, a subject having anemia due to very low, low, or intermediate risk MDS requires transfusion of 4 to 6 red blood cell units over a 8-weeks period. In certain embodiments, a subject having anemia due to very low, low, or intermediate risk MDS requires transfusion of less than 6 red blood cell units over a 8-weeks period. In certain embodiments, a subject having anemia due to very low, low, or intermediate risk MDS requires transfusion of more than 6 red blood cell units over a 8-weeks period. In certain embodiments, a subject having anemia due to very low, low, or intermediate risk MDS has a high transfusion burden. In certain embodiments, high transfusion burden is 12 or more red blood cell units over 24 weeks prior to treatment according to the dosage forms and methods provided herein. In certain embodiments, a subject treated in accordance with the dosage forms and methods provided herein has a low transfusion burden. In certain embodiments, the subject with a low transfusion burden treated in accordance with the dosage forms and methods provided herein requires at most 0, 1, 2, or 3 units of red blood cells per 8 weeks. In certain embodiments, a subject treated in accordance with the dosage forms and methods provided herein has a high transfusion burden. In certain embodiments, the subject with a high transfusion burden treated in accordance with the dosage forms and methods provided herein requires at least 4, 5, 6, 7, 8, 9, 10, 11, 12, or 13 units of red blood cells per 8 weeks.

In certain embodiments, a subject treated has one or more mutations in the SF3B1 gene. In certain embodiments, the one or more mutations in SF3B1 gene has been confirmed by genetic analysis. In certain embodiments, the one or more mutations is in a non-coding region. In certain embodiments, SF3B1 is the gene encoding SB3B1. In certain embodiments, the one or more mutations is in a coding region. In certain embodiments. SF3B1 is SF3B1 protein. In certain embodiments, the one or more mutations in SF3B1 protein is selected from the group consisting of E622D, R625C, H662Q, H662D, K66N, K666T, K666Q, K666E, A672D, K700E, 1704N. In certain embodiments, a subject treated in accordance with the dosage forms and methods provided herein expresses SF3B1 protein with the mutation E622D. In certain embodiments, a subject treated in accordance with the dosage forms and methods provided herein expresses SF3B1 protein with the mutation R625C. In certain embodiments, a subject treated in accordance with the dosage forms and methods provided herein expresses SF3B1 protein with the mutation H662Q. In certain embodiments, a subject treated in accordance with the dosage forms and methods provided herein expresses SF3B1 protein with the mutation H662D. In certain embodiments, a subject treated in accordance with the dosage forms and methods provided herein expresses SF3B1 protein with the mutation K66N. In certain embodiments, a subject treated in accordance with the dosage forms and methods provided herein expresses SF3B1 protein with the mutation K666T. In certain embodiments, a subject treated in accordance with the dosage forms and methods provided herein expresses SF3B1 protein with the mutation K666Q. In certain embodiments, a subject treated in accordance with the dosage forms and methods provided herein expresses SF3B1 protein with the mutation K666E. In certain embodiments, a subject treated in accordance with the dosage forms and methods provided herein expresses SF3B1 protein with the mutation A672D. In certain embodiments, a subject treated in accordance with the dosage forms and methods provided herein expresses SF3B1 with the mutation K700E. In certain embodiments, a subject treated in accordance with the dosage forms and methods provided herein expresses SF3B1 protein with the mutation 1704N. In a specific embodiment, a subject treated in accordance with the dosage forms and methods provided herein expresses SRSF2 with one or more mutations. In a specific embodiment, a subject treated in accordance with the dosage forms and methods provided herein expresses DNNMT3A with one or more mutations. In a specific embodiment, a subject treated in accordance with the dosage forms and methods provided herein expresses TET2 with one or more mutations. In a specific embodiment, a subject treated in accordance with the dosage forms and methods provided herein expresses SETBP1 with one or more mutations.

In certain embodiments, a subject treated in accordance with the dosage forms and methods provided herein (i) has anemia due to very low, low or intermediate risk MDS, (ii) at least 15% of erythroblasts in the subject are ring sideroblasts. In certain embodiments, a subject treated in accordance with the dosage forms and methods provided herein (i) has anemia due to very low, low or intermediate risk MDS, (ii) at least 5% of erythroblasts in the subject are ring sideroblasts, and (iii) expresses SF3B1 with one or more mutations.

In certain embodiments, a subject treated in accordance with the dosage forms and methods provided herein has thrombocytopenia. In certain embodiments, a subject treated in accordance with the dosage forms and methods provided herein has less than 100×10⁹ platelets per liter. In certain embodiments, a subject treated in accordance with the dosage forms and methods provided herein has 100 to 400×10⁹ platelets per liter. In certain embodiments, a subject treated in accordance with the dosage forms and methods provided herein has more than 400×10⁹ platelets per liter. In certain embodiments, a subject treated in accordance with the dosage forms and methods provided herein has neutropenia. In certain embodiments, a subject treated in accordance with the dosage forms and methods provided herein has an absolute neutrophil count of less than 1×10⁹ per liter.

In certain embodiments, a subject treated in accordance with the dosage forms and methods provided herein has less than 13,000 white blood cells per μL, less than 12,000 white blood cells per μL, less than 11,000 white blood cells per μL, less than 10,000 white blood cells per μL, less than 7,500 white blood cells per μL, or less than 500 white blood cells per μL.

In certain embodiments, hemoglobin levels in a subject treated in accordance with the dosage forms and methods provided herein are less than 10 g/dL, 9 g/dL, 8 g/dL, or 7 g/dL. In certain embodiments, hemoglobin levels in a subject treated in accordance with the dosage forms and methods provided herein are between 7 g/dL and 7.5 g/dL, between 7.5 g/dL and 8 g/dL, between 8 g/dL and 8.5 g/dL, between 8.5 g/dL and 9.0 g/dL, between 9.0 g/dL and 9.5 g/dL, or between 9.5 g/dL and 10.0 g/dL.

In certain embodiments of any of the foregoing methods, a subject can be refractory to prior Erythropoicsis-stimulating agents (ESA) treatment. In certain embodiments of any of the foregoing methods, a subject can be intolerant to prior ESA treatment. In certain embodiments of any of the foregoing methods, a subject can be ineligible to ESA treatment.

In certain embodiments of any of the foregoing methods, a subject who is refractory to prior ESA treatment can be a subject who has a non-response or response that is no longer maintained to prior ESA-containing regimen, either as single agent or combination with other agent, at any time after introduction due to intolerance or an adverse event.

In certain embodiments of any of the foregoing methods, the subject is intolerant to prior ESA treatment. In certain embodiments, the prior ESA-containing regimen, either as single agent or combination with other agent, at any time after introduction has been discontinued in the subject due to intolerance or an adverse event.

In certain embodiments of any of the foregoing methods, the subject is intolerant to prior ESA treatment. In certain embodiments, the subject has a low chance to respond to ESA treatments due to a high endogenous serum crythropoictin (EPO) level. In certain embodiments of any of the foregoing methods, the subject has not been previously treated with ESAs and has a serum EPO level >200 IU/L.

In certain embodiments, a subject treated in accordance with the dosage forms and methods provided herein has undergone prior treatment with one or more ESAs or is currently undergoing treatment with one or more ESAs. In certain embodiments, a subject treated in accordance with the dosage forms and methods provided herein does not respond to treatment with one or more ESAs. In certain embodiments, a subject treated in accordance with the dosage forms and methods provided herein is refractory to treatment with one or more ESAs. In certain embodiments, a subject treated in accordance with the dosage forms and methods provided herein becomes refractory to treatment with one or more ESAs. In certain embodiments, a subject treated in accordance with the dosage forms and methods provided herein is refractory to prior ESA treatment. In certain embodiments, a subject who is refractory to prior ESA treatment has documented non-response or response that is no longer maintained to prior ESA-containing regimen, either as single agent or combination with other agents (e.g., with G-CSF); the ESA regimen must have been either (a) recombinant human erythropoietin of greater than 40,000 IU/week for at least 8 doses or equivalent, or (b) darbepoetin alpha of greater than 500 μg once every three weeks for at least 4 doses or equivalent. In certain embodiments, a subject treated in accordance with the dosage forms and methods provided herein is intolerant to prior ESA-treatment. In certain embodiments, a subject who is intolerant to prior ESA-treatment has documented discontinuation of prior ESA-containing regimen, either as single agent or combination (e.g., with G-CSF), at any time after introduction due to intolerance or an adverse event. In certain embodiments, a subject treated in accordance with the dosage forms and methods provided herein is ESA-ineligible. In certain embodiments, a subject who is ESA-ineligible has a low chance of response to ESA based on an endogenous serum erythropoietin level of greater than 200 IU/L for subjects not previously treated with ESAs.

In certain embodiments, the subject treated in accordance with the methods described herein has MDS. In certain embodiments, the subject treated in accordance with the methods described herein has MDS and intact chromosome 5q. In certain embodiments, the subject treated in accordance with the dosage forms and methods provided herein has MDS, intact chromosome 5q, and does not have documented treatment failure with lenalidomide. In certain embodiments, the subject treated in accordance with the dosage forms and methods provided herein has MDS, intact chromosome 5q, and documented treatment failure with lenalidomide. In certain embodiments, the subject treated in accordance with the methods described herein has MDS with chromosome 5q deletion. MDS with chromosome 5q deletion comprises a deletion of the long arm of chromosome 5 and is characterized by, inter alia, macrocytic anemia with oval macrocytes, normal to slightly reduced white blood cell counts, normal to elevated platelet counts, and less than 5% blasts in the bone marrow and blood. In certain embodiments, the subject treated in accordance with the dosage forms and methods provided herein has MDS with chromosome 5q deletion and does not have documented treatment failure with lenalidomide. In certain embodiments, the subject treated in accordance with the dosage forms and methods provided herein has MDS with chromosome 5q deletion and documented treatment failure with lenalidomide. In certain embodiments, treatment failure with lenalidomide comprises loss of response to lenalidomide, no response to lenalidomide after 4 months of treatment with lenalidomide, intolerance to treatment with lenalidomide, or cytopenia precluding treatment with lenalidomide.

In certain embodiments, a subject treated in accordance with the dosage forms and methods provided herein has an EPO serum concentration of greater than 500 IU/L. In certain embodiments, a subject treated in accordance with the dosage forms and methods provided herein has an EPO serum concentration between 200 and 500 IU/L. In certain embodiments, a subject treated in accordance with the dosage forms and methods provided herein has an EPO serum concentration between 100 and 200 IU/L. In certain embodiments, a subject treated in accordance with the dosage forms and methods provided herein has an EPO serum concentration less than 100 IU/L.

In certain embodiments, a subject treated in accordance with the dosage forms and methods provided herein has a renal creatinine clearance rate between 40-60 mL/min. In certain embodiments, a subject treated in accordance with the dosage forms and methods provided herein has a renal creatinine clearance rate greater than 60 mL/min.

In certain embodiments, a subject treated in accordance with the dosage forms and methods provided herein has a baseline platelet count less than 100×10⁹ count/L. In certain embodiments, a subject treated in accordance with the dosage forms and methods provided herein has a baseline platelet count between 100 to 400×10⁹ count/L. In certain embodiments, a subject treated in accordance with the dosage forms and methods provided herein has a baseline platelet count greater than 400×10⁹ count/L.

In certain embodiments, a subject treated in accordance with the methods provided herein has received initial diagnosis of MDS between 0 to 2 years prior to the administration of a polypeptide comprising an amino acid sequence of SEQ ID NO: 2 or SEQ ID NO: 3. In certain embodiments, a subject treated in accordance with the dosage forms and methods provided herein has received initial diagnosis of MDS between 2 to 5 years prior to the administration of a polypeptide comprising an amino acid sequence of SEQ ID NO: 2 or SEQ ID NO: 3. In certain embodiments, a subject treated in accordance with the dosage forms and methods provided herein has received initial diagnosis of MDS more than 5 years prior to the administration of a polypeptide comprising an amino acid sequence of SEQ ID NO: 2 or SEQ ID NO: 3.

### EXEMPLIFICATION

The disclosure now being generally described, it will be more readily understood by reference to the following examples, which are included merely for purposes of illustration of certain embodiments of the present disclosure, and are not intended to limit the disclosure.

### Example 1. Generation of a ActRIIB Ligand Trap

Applicants constructed an ActRIIB Ligand Trap as follows. A polypeptide having a modified extracellular domain of ActRIIB with greatly reduced activin A binding relative to GDF11 and/or myostatin (as a consequence of a leucine-to-aspartate substitution at position 79 in SEQ ID NO: 1) was fused to a human or mouse Fe domain with a minimal linker (three glycine amino acids) in between. An ActRIIB Ligand Trap with truncated ActRIIB extracellular domain, referred to as ActRIIB(L79D 25-131)-hFc, was generated by N-terminal fusion of a TPA leader to a truncated extracellular domain (residues 25-131 in SEQ ID NO: 1) containing a leucine-to-aspartate substitution (at residue 79 in SEQ ID NO: 1) and C-terminal fusion of human Fe domain with minimal linker (three glycine residues) The ActRIIB-derived portion of the ActRIIB Ligand Trap has an amino acid sequence set forth below (SEQ ID NO: 3), and that portion could be used as a monomer or as a non-Fc fusion protein as a monomer, dimer or greater order complex.

The ActRIIB Ligand Trap protein was expressed in CHO cell lines. Three different leader sequences were considered:

(i) Honey bee melittin (HBML):
(SEQ ID NO: 5)
MKFLVNVALVFMVVYISYIYA
(ii) Tissue Plasminogen Activator (TPA):
(SEQ ID NO: 6)
MDAMKRGLCCVLLLCGAVFVSP
(iii) Native:
(SEQ ID NO: 7)
MTAPWVALALLWGSLCAGS

The selected form employs the TPA leader sequence.

To generate ActRIIB(L79D 25-131)-hFc, the human ActRIIB extracellular domain with an aspartate substitution at native position 79 (SEQ ID NO: 1) and with N-terminal and C-terminal truncations (residues 25-131 in SEQ ID NO: 1) was fused N-terminally with a TPA leader sequence instead of the native ActRIIB leader and C-terminally with a human Fc domain via a minimal linker (three glycine residues). SEQ ID NO: 4 is a nucleotide sequences encoding this fusion protein. One of ordinary skill in the art would appreciate that the expressed polypeptide would be subject to various forms of post-translational modification when expressed in cells.

Purification could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. In an example of a purification scheme, the cell culture medium is passed over a protein A column, washed in 150 mM Tris/NaCl (pH 8.0), then washed in 50 mM Tris/NaCl (pH 8.0) and eluted with 0.1 M glycine, pH 3.0. The low pH eluate is kept at room temperature for 30 minutes as a viral clearance step. The eluate is then neutralized and passed over a Q sepharose ion exchange column and washed in 50 mM Tris pH 8.0, 50 mM NaCl, and eluted in 50 mM Tris pH 8.0, with an NaCl concentration between 150 mM and 300 mM. The eluate is then changed into 50 mM Tris pH 8.0, 1.1 M ammonium sulfate and passed over a phenyl sepharose column, washed, and eluted in 50 mM Tris pH 8.0 with ammonium sulfate between 150 and 300 mM. The eluate is dialyzed and filtered for use.

Additional ActRIIB Ligand Traps (ActRIIB-Fc fusion proteins modified so as to reduce the ratio of activin A binding relative to myostatin or GDF11) are described in PCT/US2008/001506 and WO 2006/012627, incorporated by reference herein.

### Example 2: Preparation of a Lyophilized ActRIIB Ligand Trap Composition

The ActRIIB ligand trap as prepared according to Example 1 is provided as a homodimer in a composition comprising a lyophilized powder in a vial. The composition may be reconstituted with sterile water for injection. The composition is provided in 2 vial strengths, and when reconstituted with the defined quantity of Sterile Water for Injection (SWFI), each composition contains 50 mg/mL of the ActRIIB ligand trap (active pharmaceutical ingredient), and the following excipients: 10 mM citrate, 9% (w/v) sucrose, and 0.02% (w/v) polysorbate 80 at pH 6.5.

The components present in the lyophilized ActRIIB Ligand Trap composition are as follows:

| | | Lyophilized Cake | |
|---|---|---|---|
| | | (Mg per Vial) | |
| | | 25 mg/ | 75 mg/ |
| Component | Function | vial | vial |
| ActRIIB Ligand Trap | Active ingredient | 37.5 | 87.5 |
| Citric Acid monohydrate | Buffer | 0.127 | 0.296 |
| Tri-sodium citrate dehydrate | Buffer | 2.029 | 4.734 |
| Polysorbate 80 | Surfactant | 0.15 | 0.35 |
| Sucrose | Tonicity adjustment | 67.5 | 157.5 |

The lyophilized ActRIIB ligand trap composition may optionally additionally include HCl and/or NaOH added during production to adjust pH.

For administration, the lyophilate in the 25 mg vial is reconstituted with 0.68 mL Water For Injection (WFI), and the lyophilate in the 75 mg vial is reconstituted with 1.6 mL WFI. Both result in a ActRIIB ligand trap solution of at least 50 mg/mL.

### Sequences

TABLE 1
Sequences

| SEQ ID | | | Description |
|---|---|---|---|
| NO | Sequence | | |
| 1 | 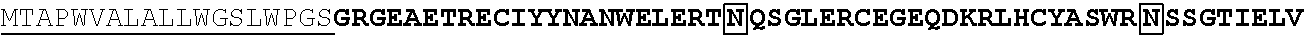 | | human |
| | KKGCWLDDFNCYDRQECVATEENPQVYFCCCEGNFCNERFTHLPEAGGPEVTYEPPPTAPTLLTVLAYSLLPI | | ActRIIB |
| | GGLSLIVLLAFWMYRHRKPPYGHVDIHEDPGPPPPSPLVGLKPLQLLEIKARGRFGCVWKAQLMNDFVAVKIF | | precursor |
| | PLQDKQSWQSEREIFSTPGMKHENLLQFIAAEKRGSNLEVELWLITAFHDKGSLTDYLKGNIITWNELCHVAE | | protein |
| | TMSRGLSYLHEDVPWCRGEGHKPSIAHRDFKSKNVLLKSDLTAVLADFGLAVRFEPGKPPGDTHGQVGTRRYM | | sequence |
| | APEVLEGAINFQRDAFLRIDMYAMGLVLWELVSRCKAADGPVDEYMLPFEEEIGQHPSLEELQEVVVHKKMRP | | |
| | TIKDHWLKHPGLAQLCVTIEECWDHDAEARLSAGCVEERVSLIRRSVNGTTSDCLVSLVTSVTNVDLPPKESS | | |
| | I | | |
| 2 | GRGEAETRECIYYNANWELERTNQSGLERCEGEQDKRLHCYASWRNSSGTIELVKKGCWLDDFNCYDRQECVA | | human |
| | TEENPQVYFCCCEGNFCNERETHLPEAGGPEVTYEPPPTAPT | | ActRIIB |
| | | | soluble |
| | | | (extracellular), |
| | | | processed |
| | | | polypeptide |
| | | | sequence |
| 3 | ETRECIYYNANWELERTNQSGLERCEGEQDKRLHCYASWRNSSGTIELVKKGCWDDDFNC | | Amino acid |
| | YDRQECVATEENPQVYFCCCEGNFCNERFTHLPEAGGPEVTYBPPPTGGGTHTCPPCPAP | | sequence of |
| | ELLGGPSVFLFPPKPKDTLMISRTPEVTCVVVDVSHEDPEVKFNWYVDGVEVHNAKTKPR | | an ActRIIB |
| | EEQYNSTYRVVSVLTVLHQDWLNGKEYKCKVSNKALPAPIEKTISKAKGQPREPQVYTLP | | ligand trap |
| | PSREEMTKNQVSLTCLVKGFYPSDIAVEWESNGQPENNYKTTPPVLDSDGSFELYSKLTV | | |
| | DKSRWQQGNVFSCSVMHEALHNHYTQKSLSLSPGK | | |
| 4 | atggatgcaa tgaagagagg gctctgctgt gtgctgctgc tgtgtggagc agtcttcgtt | 60 | Nucleotide |
| | tcgcccggcg ccgct gag aca cgg gag tgc atc tac tac aac gcc aac tgg | 111 | sequence |
| | Glu Thr Arg Glu Cys Ile Tyr Tyr Asn Ala Asn Trp | | encoding an |
| | 1 5 10 | | ActRIIB |
| | gag ctg gag cgc acc aac cag agc ggc ctg gag cgc tgc gaa ggc gag | 159 | Ligand Trap |
| | Glu Leu Glu Arg Thr Asn Gin Ser Gly Leu Glu Arg Cys Glu Gly Glu | | |
| | 15 20 25 | | |
| | cag gac aag cgg ctg cac tgc tac gcc tcc tgg cgc aac agc tct ggc | 207 | |
| | Gln Asp Lys Arg Leu His Cys Tyr Ala Ser Trp Arg Asn Ser Ser Gly | | |
| | 30 35 40 | | |
| | acc atc gag ctc gtg aag aag ggc tgc tgg gac gat gac ttc aac tgc | 255 | |
| | Thr Ile Glu Leu Val Lys Lys Gly Cys Trp Asp Asp Asp Phe Asn Cys | | |
| | 45 50 55 60 | | |
| | tac gat agg cag gag tgt gtg gcc act gag gag aac ccc cag gtg tac | 303 | |
| | Tyr Asp Arg Gln Glu Cys Val Ala Thr Glu Glu Asn Pro Gln Val Tyr | | |
| | 65 70 75 | | |
| | ttc tgc tgc tgt gaa ggc aac ttc tgc aac gag cgc ttc act cat ttg | 351 | |
| | Phe Cys Cys Cys Glu Gly Asn Phe Cys Asn Glu Arg Phe Thr His Leu | | |
| | 80 85 90 | | |
| | cca gag gct ggg ggc ccg gaa gtc acg tac gag cca ccc ccg aca | 396 | |
| | Pro Glu Ala Gly Gly Pro Glu Val Thr Tyr Glu Pro Pro Pro Thr | | |
| | 95 100 105 | | |
| | ggtggtggaa ctcacacatg cccaccgtgc ccagcacctg aactcctggg gggaccgtca | 456 | |
| | gtcttcctct tccccccaaa acccaaggac accctcatga tctcccggac ccctgaggtc | 516 | |
| | acatgcgtgg tggtggacgt gagccacgaa gaccctgagg tcaagttcaa ctggtacgtg | 576 | |
| | gacggcgtgg aggtgcataa tgccaagaca aagccgcggg aggagcagta caacagcacg | 636 | |
| | taccgtgtgg tcagcgtcct caccgtcctg caccaggact ggctgaatgg caaggagtac | 696 | |
| | aagtgcaagg tctccaacaa agccctccca gcccccatcg agaaaaccat ctccaaagcc | 756 | |
| | aaagggcagc cccgagaacc acaggtgtac accctgcccc catcccggga ggagatgacc | 816 | |
| | aagaaccagg tcagcctgac ctgcctggtc aaaggcttct atcccagcga catcgccgtg | 876 | |
| | gagtgggaga gcaatgggca gccggagaac aactacaaga ccacgcctcc cgtgctggac | 936 | |
| | tccgacggct ccttcttcct ctatagcaag ctcaccgtgg acaagagcag gtggcagcag | 996 | |
| | gggaacgtct tctcatgctc cgtgatgcat gaggctctgc acaaccacta cacgcagaag | 1056 | |
| | agcctctccc tgtccccggg taaatga | 1083 | |

| | | |
|---|---|---|
| 5 | MKFLVNVALVFMVVYISYIYA | Honey bee |
| | | melittin |
| | | (HBML) |
| | | leader |
| | | sequence |
| 6 | MDAMKRGLCCVLLLCGAVFVSP | Tissue |
| | | Plasminogen |
| | | Activator |
| | | (TPA) leader |
| | | sequence |
| 7 | MTAPWVALALLWGSLCAGS | Native |
| | | ActRIIB |
| | | leader |
| | | sequence |

The terms used in this specification generally have their ordinary meanings in the art, within the context of this disclosure and in the specific context where each term is used. Certain terms are discussed below or elsewhere in the specification, to provide additional guidance to the practitioner in describing the compositions and methods of the disclosure and how to make and use them. The scope or meaning of any use of a term will be apparent from the specific context in which the term is used.

“About” and “approximately” shall generally mean an acceptable degree of error for the quantity measured given the nature or precision of the measurements. Typically, exemplary degrees of error are within 20 percent (%), preferably within 10%, and more preferably within 5% of a given value or range of values.

Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The terms “a” and “an” include plural referents unless the context in which the term is used clearly dictates otherwise. The terms “a” (or “an”), as % well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B. and C; A, B, or C; A or C; A or B; B or C; A and C; A and B; B and C; A (alone); B (alone); and C (alone).

Numeric ranges disclosed herein are inclusive of the numbers defining the ranges.

A polypeptide disclosed herein can comprise an amino acid sequence which is not naturally occurring. Such variants necessarily have less than 100% sequence identity or similarity with the starting molecule. In certain embodiments, the variant will have an amino acid sequence from about 75% to less than 100% amino acid sequence identity or similarity with the amino acid sequence of the starting (e.g., naturally-occurring or wild-type) polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule.

Preferred methods and materials are described herein, although methods and materials similar or equivalent to those described herein can also be used in the practice or testing of the presently disclosed methods and compositions. All publications, patent applications, patents, and other references mentioned herein are incorporated by reference in their entirety.

### INCORPORATION BY REFERENCE

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the disclosure should be determined by reference to the claims, along with their full scope of equivalents, and the specification, along with such variations.

We claim:

1. A dosing regimen for the treatment of thalassemia in a subject in need thereof comprising administering a lyophilized human ActRII polypeptide linked to a constant domain of an immunoglobulin, wherein the dosing regimen comprises: 1) administering an initial dose of 1 mg/kg; 2) monitoring a subject's response; and 3) modifying the subsequent dose; and wherein the subject is administered the subsequent dose every three weeks.

2. A dosing regimen for the treatment of myelodysplastic syndrome in a subject in need thereof comprising administering a lyophilized human ActRII polypeptide linked to a constant domain of an immunoglobulin, wherein the dosing regimen comprises: 1) administering an initial dose of 1 mg/kg; 2) monitoring a subject's response; and 3) modifying the subsequent dose; and wherein the subject is administered the subsequent dose every three weeks.

3. The dosing regimen of claim 1 or 2, wherein, the subsequent dose is modified based on the subject's response.

4. The dosing regimen of any one of claims 1-3, wherein the subsequent dose is modified based on the subject's response, and wherein the subject's response is a change in red blood cell transfusion burden.

5. The dosing regimen of claim 4, wherein the subsequent dose is modified based on the subject's red blood cell transfusion burden after at least two consecutive doses.

6. The dosing regimen of any one of claim 1 or 3-5, wherein the dosing regimen is for the treatment of thalassemia, and wherein the subsequent dose is increased to 1.25 mg/kg.

7. The dosing regimen of claim 6, wherein the subsequent dose is increased to 1.25 mg/kg in a subject with no reduction in red blood cell transfusion burden.

8. The dosing regimen of any one of claim 2 or 3-5, wherein the dosing regimen is for the treatment of myelodysplastic syndrome, and wherein the subsequent dose is increased to 1.33 mg/kg or 1.75 mg/kg.

9. The dosing regimen of claim 8, wherein the subsequent dose is increased to 1.33 mg/kg or 1.75 mg/kg in a subject with no reduction in red blood cell transfusion burden.

10. The dosing regimen of any one of claims 1-9, wherein the subsequent dose is interrupted or discontinued.

11. The dosing regimen of any one of claims 1-10, wherein the subsequent dose is discontinued in a subject with no reduction in transfusion burden after three consecutive doses.

12. The dosing regimen of any one of claims 1-3, wherein the subsequent dose is modified based on the subject's response, and wherein the subject's response is a change the subject's pre-dose hemoglobin levels.

13. The dosing regimen of claim 12, wherein the subject has a pre-dose hemoglobin level greater than or equal to 11.5 g/dL in the absence of red blood cell transfusions.

14. The dosing regimen of claim 12 or 13, wherein, the subsequent dose is interrupted or discontinued.

15. The dosing regimen of claim 14, wherein, the subject's pre-dose hemoglobin levels increase greater than 2 g/dL in the absence of red blood cell transfusions, and wherein the increase occurs within three weeks of administration.

16. The dosing regimen of any one of claims 12-15, wherein, the subsequent dose is reduced.

17. The dosing regimen of any one of claims 12-16, wherein the subsequent dose is reduced to 1.33 mg/kg, 1.0 mg/kg, 0.8 mg/kg, 0.6 mg/kg, or discontinued.

18. The dosing regimen of claim 1-3, wherein, the subsequent dose is modified if the subject experiences a grade 3 or higher adverse reaction.

19. The dosing regimen of claim 18, wherein, the dose is interrupted or discontinued if the subject experiences a grade 3 or higher adverse reaction.

20. The dosing regimen of any one of claim 1 or 3-19, wherein the dosing regimen is administered to a subject with β-thalassemia.

21. The dosing regimen of any one of claim 1 or 3-19, wherein the dosing regimen is administered to a subject with α-thalassemia.

22. The dosing regimen of any one of claim 2 or 3-19, wherein the subject has very low to intermediate-risk myelodysplastic syndrome with ring sideroblasts (MDS-RS).

23. The dosing regimen of any one of claim 2 or 3-19, wherein the subject has myclodysplastic or mycloproliferative neoplasm with ring sideroblasts.

24. The dosing regimen of any one of claim 2 or 3-19, wherein the subject has thrombocytosis.

25. The dosing regimen of any one of claims 20-24, wherein the subject experiences a reduction in red blood cell transfusion burden.

26. The dosing regimen of any one of claims 20-25, wherein the subject experiences a reduction in red blood cell transfusion burden for at least 12 consecutive weeks.

27. The dosing regimen of any one of claims 20-26, wherein the subject experiences a 33% or greater reduction in red blood cell transfusion burden relative to the subject's baseline transfusion burden.

28. The dosing regimen of any one of claims 20-27, wherein the subject experiences a 50% or greater reduction in red blood cell transfusion burden relative to the subject's baseline transfusion burden.

29. The dosing regimen of any one of claims 20-28, wherein the subject becomes red blood cell transfusion independent.

30. The dosing regimen of any one of claims 20-30, wherein the subject becomes red blood cell transfusion independent for at least eight consecutive weeks.

31. The dosing regimen of any one of claims 20-30, wherein the subject becomes red blood cell transfusion independent for at least twelve consecutive weeks.

32. The dosing regimen of any one of claims claim 1-31, wherein the polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 3.

33. The dosing regimen of any one of claims 1-31, wherein the polypeptide consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 3.

34. The dosing regimen of any one of claims 1-33, wherein the polypeptide is part of a homodimer protein complex.

35. The dosing regimen of any one of claims 1-34, wherein the initial dose or subsequent dose is administered parenterally.

36. The dosing regimen of any one of claims 1-35, wherein the initial dose or subsequent dose is administered via subcutaneous injection.

37. The dosing regimen of any one of claims 1-36, wherein the polypeptide is provided as a lyophilized powder in a vial.

38. The dosing regimen of claim 37, wherein the lyophilized powder is provided in an amount of 25 mg/vial or 75 mg/vial.

39. The dosing regimen of claim 37 or 38, wherein the lyophilized powder is reconstituted with sterile water for injection.

40. The dosing regimen of any one of claims 37-39, wherein the lyophilized powder is reconstituted with Sterile Water for Injection to a final polypeptide concentration of approximately 45 mg/mL, 46 mg/mL, 47 mg/mL, 48 mg/mL, 49 mg/mL, 50 mg/mL, 51 mg/mL, 52 mg/mL, 53 mg/mL, 54 mg/mL, or 55 mg/mL.

41. The dosing regimen of any one of claims 37-40, wherein the lyophilized powder is reconstituted with Sterile Water for Injection to a final polypeptide concentration of approximately 50 mg/mL.

42. The dosing regimen of any one of claims 37-41, wherein the lyophilized powder is reconstituted with approximately 0.5 mL, 0.56 mL, 0.58 mL, 0.6 mL, 0.62 mL, 0.64 mL, 0.66 mL, 0.68 mL, 0.70 mL, 0.72 mL, 0.74 mL, 0.76 mL, 0.78 mL, 0.8 mL, 0.82 mL, 0.84 mL, 0.86 mL, 0.88 mL, 0.9 mL, 0.92 mL, 0.94 mL, 0.96 mL, 0.98 mL, 1 mL, 1.1 mL, 1.15 mL, 1.2 mL, 1.25 mL, 1.3 mL, 1.35 mL, 1.4 mL, 1.45 mL, 1.5 mL, 1.55 mL, 1.6 mL, 1.65 mL, 1.7 mL, 1.75 mL, or 1.8 mL of Sterile Water for Injection.

43. The dosing regimen of any one of claims 37-41, wherein the lyophilized powder for injection is provided in an amount of 25 mg/vial, and wherein the polypeptide is reconstituted with 0.65 mL, 0.66 mL, 0.67 mL, 0.68 mL, 0.69 mL, 0.70 mL, 0.71 mL, 0.72 mL, 0.73 mL, 0.74 mL, or 0.75 mL of Sterile Water for Injection.

44. The dosing regimen of any one of claims 37-43, wherein the lyophilized powder for injection is provided in an amount of 75 mg/vial, and wherein the polypeptide is reconstituted with 0.65 mL, 0.66 mL, 1.55 mL, 1.56 mL, 1.57 mL, 1.58 mL, 1.59 mL, 1.6 mL, 1.61 mL, 1.62 mL, 1.63 mL, 1.64 mL, 1.65 mL, 1.66 mL, 1.67 mL, 1.68 mL, 1.69 mL, 1.7 mL, 1.71 mL, 1.72 mL, 1.73 mL, 1.74 mL, or 1.75 mL of Sterile Water for injection.

45. The dosing regimen of any one of claims 37-44, wherein the vial comprises a lyophilized powder and one or more pharmaceutical additives and/or excipients.

46. The dosing regimen of claim 45, wherein one or more of the pharmaceutical additives and/or excipients is a buffering agent.

47. The dosing regimen of claim 46, wherein the buffering agent is selected to be physiologically compatible and to maintain a pH of 5.5, 5.7, 6.0, 6.3, 6.5, 6.7, 7.0, 7.3, 7.5, 7.7, 8.0, 8.3, 8.5, 8.7, 9.0, 9.3, 9.5, 9.7, or 10.0 when reconstituted with Sterile Water for Injection.

48. The dosing regimen of claim 46 or 47, wherein the buffering agent is selected to be physiologically compatible and to maintain a pH of 6.0, 6.3, 6.5, 6.7, 7.0, 7.3, or 7.5 when reconstituted with Sterile Water for Injection.

49. The dosing regimen of any one of claims 46-48, wherein the buffering agent is selected to be physiologically compatible and to maintain a pH of 6.5 when reconstituted with Sterile Water for Injection.

50. The dosing regimen of any one of claims 46-49, wherein the buffering agent comprises organic acids, succinate, phosphate, acetate, citrate, citric acid, Tris, HEPES, amino acids, or mixtures of amino acids.

51. The dosing regimen of any one of claims 46-50, wherein the buffering agent comprises tri-sodium citrate dihydrate.

52. The dosing regimen of any one of claims 46-51, wherein the buffering agent comprises citric acid monohydrate.

53. The dosing regimen of any one of claims 46-52, wherein the buffering agent comprises tri-sodium citrate dihydrate and citric acid monohydrate.

54. The dosing regimen of any one of claims 46-53, wherein the buffering agent comprises a concentration of at least 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 25, 30, 40, 50, 60, 70, 80, 90, 100, 200, or 500 mM.

55. The dosing regimen of any one of claims 46-54, wherein the buffering agent comprises a concentration of at least 10 mM.

56. The dosing regimen of any one of claims 45-55, wherein one or more of the pharmaceutical additives and/or excipients is a stabilizer.

57. The dosing regimen of claim 56, wherein the stabilizer is selected from the group consisting of: sucrose, trehalose, mannose, maltose, lactose, glucose, raffinose, cellobiose, gentiobiose, isomaltose, arabinose, glucosamine, fructose, mannitol, sorbitol, poly-hydroxy compounds, polysaccharides, dextran, starch, hydroxyethyl starch, cyclodextrins, N-methyl pyrollidene, cellulose, or hyaluronic acid.

58. The dosing regimen of claim 56 or 57, wherein the stabilizer is sucrose.

59. The dosing regimen of any one of claims 56-58, wherein the stabilizer comprises a concentration of at least 0.005% w/v, 0.01% w/v, 0.02% w/v, 0.03% w/v, 0.05% w/v, 0.06% w/v, 0.07% w/v, 0.08% w/v, 0.09% w/v, 0.1% w/v, 0.5% w/v, 0.7% w/v, 0.8% w/v, 0.9% w/v, 1.0% w/v, 1.2% v/v, 1.5% w/v, 1.7% w/v, 2% w/v, 3% w/v, 4% w/v, 5% w/v, 6% w/v, 7% w/v, 8% w/v, 9% w/v, 10% w/v, 11% w/v, 12% w/v, 13% w/v, 14% w/v, 15% w/v, 16% w/v, 17% w/v, 18% w/v, 19% w/v, or 20% w/v.

60. The dosing regimen of any one of claims 56-59, wherein the stabilizer comprises a concentration of at least 9% w/v.

61. The dosing regimen of any one of claims 45-60, wherein one or more of the pharmaceutical additives and/or excipients is a surfactant.

62. The dosing regimen of claim 61, wherein the surfactant is selected from the group consisting of: sodium lauryl sulfate, dioctyl sodium sulfosuccinate and dioctyl sodium sulfonate, chenodeoxycholic acid, N-lauroylsarcosine sodium salt, lithium dodecyl sulfate, 1-octanesulfonic acid sodium salt, sodium cholate hydrate, sodium deoxycholate, and glycodeoxycholic acid sodium salt, benzalkonium chloride, benzethonium chloride, cetylpyridinium chloride monohydrate, hexadecyltrimethylammonium bromide, CHAPS, CHAPSO, SB3-10, SB3-12, digitonin, Triton X-100, Triton X-114, TWEEN-20, TWEEN-80, lauromacrogol 400, polyoxyl 40 stearate, polyoxyethylene hydrogenated castor oil 10, 40, 50 and 60, glycerol monostearate, polysorbate 40, polysorbate 60, polysorbate 65, polysorbate 80, or soy lecithin.

63. The dosing regimen of claim 61 or 62, wherein the surfactant is polysorbate 80.

64. The dosing regimen of any one of claims 61-63, wherein the surfactant comprises a concentration of at least 0.001, 0.002, 0.003, 0.004, 0.005, 0.01, 0.02, 0.03, 0.05, 0.06, 0.07, 0.08, 0.09, 0.1, 0.5, 0.7, 0.8 0.9, or 1.0% w/v.

65. The dosing regimen of any one of claims 61-64, wherein the surfactant comprises a concentration of at least 0.2% w/v.

66. The dosing regimen of any one of claims 37-65, wherein the vial comprises a lyophilized powder comprising the polypeptide, citric acid monohydrate, tri-sodium citrate dehydrate, polysorbate 80, and sucrose.

67. The dosing regimen of any one of claims 37-66, wherein the vial comprises a lyophilized powder comprising 37.5 mg of the polypeptide, 0.127 mg citric acid monohydrate, 2.029 mg tri-sodium citrate dehydrate, 0.15 mg polysorbate 80, and 67.5 mg sucrose.

68. The dosing regimen of any one of claims 37-66, wherein the vial comprises a lyophilized powder comprising 87.5 mg ActRII polypeptide, 0.296 mg citric acid monohydrate, 4.734 mg tri-sodium citrate dehydrate, 0.35 mg polysorbate 80, and 157.5 mg sucrose.


SEQUENCE LISTING (8 sequences)

SEQ ID NO: 1 - protein, 512 aa, Homo sapiens
MTAPWVALALLWGSLWPGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 2 - protein, 115 aa, Homo sapiens
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 3 - protein, 335 aa, Artificial Sequence
  Description of Artificial Sequence Synthetic polypeptide
ETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 1083 bp, Artificial Sequence
  Description of Artificial Sequence Synthetic polynucleotide
atggatgcaatgaagagagggctctgctgtgtgctgctgctgtgtggagcagtcttcgtttcgcccggcgccgctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctgggacgatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacaggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 5 - protein, 21 aa, Apis sp.
MKFLVNVALVFMVVYISYIYA

SEQ ID NO: 6 - protein, 22 aa, Unknown
  Description of Unknown Tissue plasminogen activator sequence
MDAMKRGLCCVLLLCGAVFVSP

SEQ ID NO: 7 - protein, 19 aa, Homo sapiens
MTAPWVALALLWGSLCAGS

SEQ ID NO: 8 - protein, 107 aa, Artificial Sequence
  Description of Artificial Sequence Synthetic polypeptide
ETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPT